(19) United States
(12) Patent Application Publication
(10) Pub. No.: US 2026/0321353 A1
(43) Pub. Date: Oct. 15, 2026

(54) METHODS AND SYSTEMS FOR SELECTING FEATURES AND USES THEREOF

(71) Applicant: Foundation Medicine, Inc., Boston, MA (US)

(72) Inventors: Haleh ASHKI, Escondido, CA (US); Ray Abrams GREENSTEIN, West Hartford, CT (US); Mark R. KENNEDY, Cambridge, MA (US)

(73) Assignee: Foundation Medicine, Inc., Boston, MA (US)

(21) Appl. No.: 19/644,656

(22) Filed: Apr. 10, 2026

Related U.S. Application Data
(60) Provisional application No. 63/787,546, filed on Apr. 11, 2025.

(51) Int. Cl. G16B 40/20; G06N 20/00

(52) U.S. Cl. CPC G16B 40/20; G06N 20/00

(57) ABSTRACT

Methods for generating a feature group for distinguishing a first disease characterization class from a second disease characterization class are described. The methods may comprise, for example, selecting pairwise subclass representative features that distinguish disease characterization subclasses from other disease characterization; selecting a first subset of the plurality of the disease characterization subclass labels as belonging to the first disease characterization class and a second subset of the plurality of the disease characterization subclass labels as belonging to the second disease characterization class; selecting a subset of pairwise subclass representative features from the pairwise subclass representative features, wherein the subset of pairwise subclass representative features distinguish disease characterization subclasses from within a common disease characterization class; generating a feature group by removing the subset of pairwise subclass representative features from the pairwise subclass representative features.

### CROSS-REFERENCE TO RELATED APPLICATIONS

This application claims priority benefit of U.S. Provisional Patent Application Ser. No. 63/787,546, filed Apr. 11, 2025, the contents of which are incorporated herein by reference in their entirety.

### FIELD

The present disclosure relates generally to methods and systems for analyzing genomic profiling data, and more specifically to methods and systems for generating a feature group for distinguishing diseases using genomic profiling data. Also described are methods of designing and/or making a plurality of nucleic acid bates (i.e., a bait set) that covers one of more features of the feature group for selective enrichment.

### BACKGROUND

Blood-based analysis has great promise as a tool for early detection of cancer in patients using a minimally invasive clinical sample. But a challenge in the field is to identify features that are informative across different disease ontologies, sub-classes and stages of development. Often methods fail to incorporate biological insight into feature selection processes and thus unreliable features are used for designing clinical tests. Uninformed feature selection may lead to overfitting of models especially when there is significant variation within training sample, such as in genomic cancer profiles. The specificity of informative features for early detection of conditions is particularly important.

### BRIEF SUMMARY OF THE INVENTION

Disclosed herein are methods and systems for generating a feature group for distinguishing a first disease characterization class from a second disease characterization class. The first and second disease characterization class may comprise cancer and non-cancer disease characterizations respectfully and thus the methods and systems can be used to generate feature groups for distinguishing samples with cancer from samples without cancer. The features can be used to generate nucleic acid bait sets and for training machine learning models to effectively distinguish between the first disease characterization class and second disease characterization class.

The methods and systems utilize biological informed subclasses to improve feature selection between classes. The feature groups generated using the methods described herein may comprise genomic regions that can be used to generate biologically informed nucleic acid bait sets for targeted genomic sequencing. Targeted genomic sequencing allows for higher coverage sequencing of informative regions without increasing the overall sequencing depth. The feature groups generated using the methods described herein can also be used to train machine learning models, such as machine learning models trained to classify samples as belonging to the first disease characterization subclass or second disease characterization subclass. The machine learning models will perform better when trained with biologically informed features.

Provided herein are methods of generating a feature group for distinguishing a first disease characterization class from a second disease characterization class, comprising: receiving, at one or more processors, training data comprising values for features for a plurality of training samples comprising a disease characterization subclass label, wherein the disease characterization subclass label represents one of a plurality of disease characterization subclasses to which the training sample belongs, and wherein the plurality of training samples represents a plurality of disease characterization subclass labels; selecting, by the one or more processors, pairwise subclass representative features that distinguish the disease characterization subclasses from other disease characterization subclasses in the plurality of disease characterization subclasses; selecting, by one or more processors, a first subset of the plurality of the disease characterization subclass labels as belonging to the first disease characterization class and a second subset of the plurality of the disease characterization subclass labels as belonging to the second disease characterization class; selecting, by one or more processors, a subset of pairwise subclass representative features from the pairwise subclass representative features, wherein the subset of pairwise subclass representative features that distinguish disease characterization subclasses from within a common disease characterization class; generating, by the one or more processors, a feature group by removing the subset of pairwise subclass representative features from the pairwise subclass representative features.

In some aspects, the features are associated with a plurality of subject intervals, a plurality of genes, a plurality of epigenetic states and/or a plurality of genetic alterations. In some aspects, the values comprise mRNA expression values, protein expression values, methylation levels, and/or a genetic alteration status.

In some aspects, the features are associated with a plurality of microbacterium species. In some aspects, the values comprise an abundance of a microbacterium species in the plurality of microbacterium species.

In some aspects, the disease characterizing subclasses relate to a pathological status, tissue of origin, or stage of a disease. In some aspects, the first disease characterization class and/or second characterization class comprises a cancer characterization. In some aspects, the first disease characterization class is cancer, and the second disease characterization class is non-cancer. In some aspects, the first subset of disease characterization subclasses comprises, colorectal cancer, advanced adenoma, lung cancer, lung adenocarcinoma, lung squamous-cell carcinoma, esophageal cancer, stomach cancer, liver cancer, bile duct cancer, pancreatic cancer, bladder cancer, and ovarian cancer, and the second subset of disease characterization subclasses comprises non-advanced adenoma and non-cancer. In some aspects, the first disease characterization class is colorectal cancer, and the second disease characterization class is lung cancer. In some aspects, the first subset of disease characterization subclasses comprises, colorectal cancer stage I, colorectal cancer stage II, colorectal cancer stage III, colorectal cancer stage IV, and the second subset of disease characterization subclasses comprises lung cancer stage I, lung cancer stage II, lung cancer stage III, lung cancer stage IV.

In some aspects, selecting the pairwise subclass representative feature comprises use of one or more of an information gain model, Random forest model, Anova model, correlation filtering model, or T-test model.

Also provided herein are methods of designing a plurality of nucleic acid baits, comprising: generating a feature group according to any one of the methods described herein; designing a plurality of nucleic acid baits corresponding to genomic regions covering one or more features of the feature group.

Also provided herein are methods of making a plurality of nucleic acid baits, comprising designing a feature group according to the methods described herein; making the plurality of nucleic acid baits corresponding to genomic regions covering one or more features of the feature group.

Also provided herein are methods of training a machine learning model to classify a test sample as a first disease characterization class or a second characterization class, comprising: receiving, at one or more processors, training data comprising values for features for a plurality of training samples, comprising a disease characterization subclass label, wherein the disease characterization subclass label represents one of a plurality of disease characterization subclasses the training sample belongs to and wherein the plurality of training samples represents a plurality of disease characterization subclass labels, and wherein the features comprise a plurality of features from a feature group generated according to the methods described herein; selecting, by one or more processors, a first subset of the plurality of the disease characterization subclass labels as belonging to the first disease characterization class and a second subset of the plurality of the disease characterization subclass labels as belonging to the second disease characterization class; training, by the one or more processors a machine learning model to classify a test sample as the first disease characterization class or the second disease characterization class using the training data, and the plurality of features from the feature group.

In some aspects, the methods further comprise, performing a dimensionality reduction method on the feature group before training the machine learning model. In some aspects, the dimensionality reduction method comprises principal component analysis (PCA) or UMAP. In some aspects, the machine learning model comprises one or more of a GLMnet, Random Forest, Support Vector Machine, Naïve Boost, Naïve Bayesian, XGboost, or Logistic regression model.

Also provided herein are methods of classifying a test sample for a subject as a first disease characterization class or a second disease characterization class comprising: receiving, at one or more processors, test data from a test sample collected from the subject, test data comprising values for a plurality of features of a feature group generated according to the methods described herein inputting, by the one or more processors, the test data into a machine learning model trained to classify a test sample as the first disease characterization class or the second disease characterization class according to the method described herein classifying the test sample as the first disease characterization class or the second disease characterization class based on the output from the classification model.

Also provided herein are methods comprising: providing a plurality of nucleic acid molecules obtained from a test sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, using one or more processors, sequence read data for the plurality of sequence reads; generating, using the one or more processors, test data from the sequence read data, wherein the test data comprises values for features; inputting, by the one or more processors, the test data into a machine learning model trained to classify a test sample as the first disease characterization class or the second disease characterization class according to the methods described herein, classifying the test sample as the first disease characterization class or the second disease characterization class based on the output from the classification model.

In some aspects, the training data has been generated by: providing a plurality of nucleic acid molecules obtained from a training sample of the plurality of training samples; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, using one or more processors, sequence read data for the plurality of sequence reads; generating, using the one or more processors, training data from the sequence read data, wherein the training data comprises values for features.

In some aspects, the subject is suspected of having or is determined to have cancer. In some aspects, the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor. In some aspects, cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

In some aspects, the methods further comprise treating the subject with an anti-cancer therapy. In some aspects, the anti-cancer therapy comprises a targeted anti-cancer therapy. In some aspects, the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some aspects, the methods further comprise obtaining the plurality of training samples from a plurality of training subjects.

In some aspects, the methods further comprise obtaining the test sample from the subject.

In some aspects, the plurality of training samples comprises a tissue biopsy sample, a liquid biopsy sample, or a normal control.

In some aspects, the test sample comprises a tissue biopsy sample, a liquid biopsy sample, or a normal control. In some aspects, the test sample and/or the plurality of training samples is a liquid biopsy sample and comprises blood, plasma, cerebrospinal fluid, sputum, stool, urine, or saliva. In some aspects, the test sample and/or the plurality of training samples is a liquid biopsy sample and comprises circulating tumor cells (CTCs). In some aspects, the test sample and/or plurality of training samples is a liquid biopsy sample and comprises cell-free DNA (cfDNA). In some aspects, the cell-free DNA (cfDNA) or a portion thereof comprises circulating tumor DNA (ctDNA). In some aspects, the plurality of nucleic acid molecules comprises a mixture of tumor nucleic acid molecules and non-tumor nucleic acid molecules. In some aspects, the tumor nucleic acid molecules are derived from a tumor portion of a heterogeneous tissue biopsy sample, and the non-tumor nucleic acid molecules are derived from a normal portion of the heterogeneous tissue biopsy sample. In some aspects, the test sample and/or plurality of training samples comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

In some aspects, the one or more adapters comprise amplification primers, flow cell adaptor sequences, substrate adapter sequences, or sample index sequences. In some aspects, the captured nucleic acid molecules are captured from the amplified nucleic acid molecules by hybridization to one or more bait molecules. In some aspects, the one or more bait molecules comprise one or more nucleic acid molecules, each comprising a region that is complementary to a region of a captured nucleic acid molecule. In some aspects, amplifying nucleic acid molecules comprises performing a polymerase chain reaction (PCR) amplification technique, a non-PCR amplification technique, or an isothermal amplification technique.

In some aspects, the sequencing comprises use of a massively parallel sequencing (MPS) technique, whole genome sequencing (WGS), whole exome sequencing, targeted sequencing, direct sequencing, or Sanger sequencing technique. In some aspects, the sequencing comprises massively parallel sequencing, and the massively parallel sequencing technique comprises next generation sequencing (NGS). In some aspects, the sequencer comprises a next generation sequencer.

In some aspects, the one or more of the plurality of sequencing reads overlap one or more gene loci within one or more subject intervals in the sample. In some aspects, the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci. In some aspects, the one or more gene loci comprise ABL1, ACVRIB, AKT1, AKT2, AKT3, ALK, ALOX12B, AMER1, APC, AR, ARAF, ARFRP1, ARIDIA, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKNIA, CDKNIB, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNFIA, HRAS, HSD3B1, ID3, IDH1, IDH2, IGFIR, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCDILG2, PDGFRA, PDGFRB, PDK1, PIK3C2B, PIK3C2G, PIK3CA, PIK3CB, PIK3R1, PIM1, PMS2, POLD1, POLE, PPARG, PPP2RIA, PPP2R2A, PRDM1, PRKARIA, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSC1L1, WT1, XPO1, XRCC2, ZNF217, ZNF703, or any combination thereof. In some aspects, the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

In some aspects, the methods further comprise generating, by the one or more processors, a report indicating the classification of the test sample as the first disease characterization class or the second disease characterization class.

In some aspects, the methods further comprise transmitting the report to a healthcare provider. In some aspects, the report is transmitted via a computer network or a peer-to-peer connection.

In some aspects, the classification of the test sample as the first disease characterization class or the second disease characterized class is used to diagnose or confirm a diagnosis of a disease in the subject. In some aspects, the disease is cancer. In some aspects, the cancer is colorectal cancer.

In some aspects, the methods further comprise selecting an anti-cancer therapy to administer to the subject based on the classification of the test sample.

In some aspects, the methods further comprise determining an effective amount of an anti-cancer therapy to administer to the subject based on the classification of the test sample.

In some aspects, the methods further comprise administering an anti-cancer therapy to the subject based on the classification of the test sample. In some aspects, the anti-cancer therapy comprises chemotherapy, radiation therapy, immunotherapy, a targeted therapy, or surgery.

Also provided herein are methods for diagnosing a disease, the method comprising: diagnosing that a subject has the disease based on a classification of a test sample from the subject as a first disease characterization class or a second disease characterization class, wherein the test sample is classified according to the methods described herein.

Also provided herein are methods of selecting an anti-cancer therapy, the method comprising: classifying a test sample from the subject as a first disease characterization class or a second disease characterization class according to the methods described herein; responsive to the classification of the test sample as the first disease characterization class or second disease characterization class, selecting an anti-cancer therapy for the subject.

Also provided herein are methods of treating a cancer in a subject, comprising: classifying a test sample from the subject as a first disease characterization class or a second disease characterization class according to the methods described herein; responsive to the classification of the test sample, administering an effective amount of an anti-cancer therapy to the subject.

Also provided herein are methods for monitoring cancer progression or recurrence in a subject, the method comprising: classifying a first test sample from the subject as a first disease characterization class or a second disease characterization class according to the methods described herein; determining a second classification in a second test sample obtained from the subject at a second time point; and comparing the first classification to the second classification, thereby monitoring the cancer progression or recurrence. In some aspects, the second classification for the second test sample is classified according to the methods described herein. In some aspects, the methods further comprise selecting an anti-cancer therapy for the subject in response to the cancer progression. In some aspects, the methods further comprise administering an anti-cancer therapy to the subject in response to the cancer progression. In some aspects, the methods further comprise adjusting an anti-cancer therapy for the subject in response to the cancer progression. In some aspects, the methods further comprise adjusting a dosage of the anti-cancer therapy or selecting a different anti-cancer therapy in response to the cancer progression. In some aspects, the methods further comprise administering the adjusted anti-cancer therapy to the subject.

In some aspects, the first time point is before the subject has been administered an anti-cancer therapy, and wherein the second time point is after the subject has been administered the anti-cancer therapy. In some aspects, the subject has a cancer, is at risk of having a cancer, is being routine tested for cancer, or is suspected of having a cancer.

In some aspects, wherein the cancer is a solid tumor. In some aspects, the cancer is a hematological cancer.

In some aspects, the anti-cancer therapy comprises chemotherapy, radiation therapy, immunotherapy, a targeted therapy, or surgery.

In some aspects, the methods further comprise determining, identifying, or applying the classification of the test sample from the subject as a diagnostic value associated with the test sample.

In some aspects, the methods further comprise generating a genomic profile for the subject based on the classification of the test sample. In some aspects, the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof. In some aspects, the genomic profile for the subject further comprises results from a nucleic acid sequencing-based test.

In some aspects, the methods further comprise selecting an anti-cancer therapy, administering an anti-cancer therapy, or applying an anti-cancer therapy to the subject based on the generated genomic profile.

In some aspects, the classification of the test sample as a first disease classification class and/or a second disease classification class is used in making suggested treatment decisions for the subject. In some aspects, the classification of the test sample as a first disease classification class and/or a second disease classification class for the test sample is used in applying or administering a treatment to the subject.

Also provided herein are bait sets comprising a plurality of nucleic acid baits made according to the methods described herein.

Also provided herein are systems comprising: one or more processors; and a memory communicatively coupled to the one or more processors and configured to store instructions that, when executed by the one or more processors, cause the system to: receive, at one or more processors, training data comprising values for features for a plurality of training samples comprising a disease characterization subclass label, wherein the disease characterization subclass label represents one of a plurality of disease characterization subclasses to which the training sample belongs to and wherein the plurality of training samples represents a plurality of disease characterization subclass labels; select, by the one or more processors, pairwise subclass representative features that distinguish the disease characterization subclasses from other disease characterization subclasses in the plurality of disease characterization subclasses; select, by one or more processors, a first subset of the plurality of the disease characterization subclass labels as belonging to the first disease characterization class and a second subset of the plurality of the disease characterization subclass labels as belonging to the second disease characterization class; select, by one or more processors, a subset of pairwise subclass representative features from the pairwise subclass representative features, wherein the subset of pairwise subclass representative features that distinguish disease characterization subclasses from within a common disease characterization class; generate, by the one or more processors, a feature group by removing the subset of pairwise subclass representative features from the pairwise subclass representative features.

Also provided herein are non-transitory computer-readable storage mediums storing one or more programs, the one or more programs comprising instructions, which when executed by one or more processors of a system, cause the system to: receive, at one or more processors, training data comprising values for features for a plurality of training samples comprising a disease characterization subclass label, wherein the disease characterization subclass label represents one of a plurality of disease characterization subclasses to which the training sample belongs to and wherein the plurality of training samples represents a plurality of disease characterization subclass labels; select, by the one or more processors, pairwise subclass representative features that distinguish the disease characterization subclasses from other disease characterization subclasses in the plurality of disease characterization subclasses; select, by one or more processors, a first subset of the plurality of the disease characterization subclass labels as belonging to the first disease characterization class and a second subset of the plurality of the disease characterization subclass labels as belonging to the second disease characterization class; select, by one or more processors, a subset of pairwise subclass representative features from the pairwise subclass representative features, wherein the subset of pairwise subclass representative features that distinguish disease characterization subclasses from within a common disease characterization class; generate, by the one or more processors, a feature group by removing the subset of pairwise subclass representative features from the pairwise subclass representative features.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1 provides a non-limiting example of a process for generating a feature group set and use thereof according to embodiments described herein.

FIG. 2 depicts an exemplary computing device or system in accordance with one embodiment of the present disclosure.

FIG. 3 depicts an exemplary computer system or computer network, in accordance with some instances of the systems described herein.

### DETAILED DESCRIPTION

Provided herein are methods and systems that can be used to improve cancer diagnostics and treatment selection for patients with cancer using novel machine learning approaches comparing biologically informed feature selection. The approaches described herein improve on the ability to distinguish samples as cancer or non-cancer samples, or other disease states (e.g., cancer staging), through designing and making of nucleic acid baits that improve sequencing efficiency of informative subject (e.g. subgenomic) intervals and/or identifying informative features for classification machine learning models. In some cases, it may be difficult for a pathologist to distinguish between cancer and non-cancerous tumors morphologically or with simple molecular data and thus machine learning with deeper molecular profiling is necessary.

Upon collection of a sample from a cancer patient, the features identified as informative for distinguishing cancer and non-cancerous tumors, or other disease states, can be assayed in the sample. The values for the informative features may be input to a machine learning model trained to distinguish the sample as a cancer or non-cancer, or to distinguish the sample as associated with disease state from another disease state. Using this classification, effective treatments plans can be developed for patients with cancer or other diseases. The methods rely at least on the relationship between disease characterization subclasses (types/stages of cancer and/or non-cancerous tumors) to improve the ability to design methods that can distinguish between disease characterization classes (cancer and non-cancer, or other disease stage).

In some implementations, the methods are used to distinguish different stages of cancer more effectively. Cancer stage diagnosis may be difficult and can lead to more effective treatment plans. In a non-limiting example, the methods may be used to identify feature groups that are informative for distinguishing early (stage I or stage II) and late stage (stage III or stage IV) cancer. In such embodiments, training data may be collected from samples collected from patients with stage I, stage II, stage III, or stage IV cancer. The training data may comprise methylation features at several subgenomic intervals and the values may be methylation levels at each of the subgenomic intervals for each of the samples. The disease characterization classes may be stage I, stage II, stage III, or stage IV cancer and each sample in the training sample is labelled accordingly. Using the methods described herein, the subgenomic intervals that are most informative for distinguishing early and late stage cancer can be selected. A patient can be effectively diagnosed as having early or late stage cancer and a treatment plan can be designed based on characterizations of the informative features in a sample from the patient. The methods can also be used for cancer monitoring because a sample can be collected at multiple timepoints and the informative features can be assessed at each timepoint.

Provided herein are approaches that can be used for selecting features based on biologically informed sub class labels of samples. Instead of looking for informative features across two groups of samples (test-positive and test-negative) in aggregate, the approaches rely on considerations of the distinct biological makeup of the samples in each of the cohorts. The approaches described herein leverage biological diversity and biological insights to improve features selection methods and downstream uses of the features in characterizing samples. For example, features can be annotated as informative or not for various sub-comparisons and used appropriately in downstream model architectures for classifying samples and/or for designing nucleic acid baits for enriching for informative genomic regions in sequencing. The approaches described herein allow for flexibility in feature selection for specific nucleic acid bait set design and sample classification tasks because the sub classes can be changed based on the in-group versus out-group disease classes desired by the user.

Methods and systems for generating a feature group for distinguishing a first disease characterization class from a second disease characterization class are described. The methods and systems comprise selecting disease characterization subclasses for each of the first and second disease characterization classes and using the relationship between the subclasses to inform feature selection. Pairwise feature selection is performed between each of the disease characterization subclasses and then features identified within disease characterization subclasses belonging to a common disease characterization class are removed. Pairwise feature selection followed by removal of features that are common to a disease characterization class allow for the selection of features that distinguish between the disease classes without interference of variability within the disease characterization class.

Variation between subclasses within a disease characterization class may overshadow variation distinguishing classes and prevent the identification of informative features for distinguishing the two classes. The within class variation may contribute to overfitting of models especially when there is significant variation within training samples, such as in genomic cancer profiles. Thus, the methods described herein improve upon conventional methods for training machine learning classification models for early diagnosis of disease and methods for designing nucleic acid bait sets to improve targeted sequencing efficiency.

In some instances, the methods and systems can be used for improving genomic and transcriptomic analysiss. In some instances, the features are associated with genomic intervals, genes, and/or genetic alteration, for example a genomic metric at a specific subject or subgenomic interval. The values associated with these features that may be used for feature selection include genomic and transcriptomic information about a sample such as, mRNA expression values, protein expression values, methylation levels, and/or a genetic alteration status. In some instances, the methods and systems can be used for improving microbiome analysis. In some instances, the features are associated with a plurality of microbacterium species identified in a sample and the values comprise abundance of a microbacterium.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the individual, patient, or subject herein is a human.

The terms “cancer” and “tumor” are used interchangeably herein. These terms refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell, such as a leukemia cell. These terms include a solid tumor, a soft tissue tumor, or a metastatic lesion. As used herein, the term “cancer” includes premalignant, as well as malignant cancers.

As used herein, “treatment” (and grammatical variations thereof such as “treat” or “treating”) refers to clinical intervention (e.g., administration of an anti-cancer agent or anti-cancer therapy) in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the term “subgenomic interval” (or “subgenomic sequence interval”) refers to a portion of a genomic sequence.

As used herein, the term “subject interval” refers to a subgenomic interval or an expressed subgenomic interval (e.g., the transcribed sequence of a subgenomic interval).

As used herein, the term “sequence read” is a computationally generated sequence generated by a sequencer to represent a sequence of bases of a single strand of a sequenced fragment. A sequence read can refer to a raw sequence read (e.g., a sequence read as obtained directly from a sequencing instrument), an aligned sequence read (e.g., a sequence read that has been aligned to a reference genome), a single-end sequence read, a paired-end sequence read, a merged sequence read (e.g., a sequence read based on merging a group of overlapping paired-end reads), a consensus sequence read (e.g., a sequence read based on performing error correction on a merged sequence read), a computationally reconstructed sequence read (e.g., a sequence read that has been computationally truncated at the 5′ and/or 3′ end), or any combination thereof.

As used herein, the terms “variant sequence” or “variant” are used interchangeably and refer to a modified nucleic acid sequence relative to a corresponding “normal” or “wild-type” sequence. In some instances, a variant sequence may be a “short variant sequence” (or “short variant”), i.e., a variant sequence of less than about 50 base pairs in length.

The terms “allele frequency” and “allele fraction” are used interchangeably herein and refer to the fraction of sequence reads corresponding to a particular allele relative to the total number of sequence reads for a genomic locus.

The terms “variant allele frequency” and “variant allele fraction” are used interchangeably herein and refer to the fraction of sequence reads corresponding to a particular variant allele relative to the total number of sequence reads for a genomic locus.

The section headings used herein are for organizational purposes only and are not to be construed as limiting the subject matter described.

The figures illustrate processes according to various embodiments. In the exemplary processes, some blocks are, optionally, combined, the order of some blocks is, optionally, changed, and some blocks are, optionally, omitted. In some examples, additional steps may be performed in combination with the exemplary processes. Accordingly, the operations as illustrated (and described in greater detail below) are exemplary by nature and, as such, should not be viewed as limiting.

Finally, it is to be understood that features and preferences described in relation to “embodiments” are distinct preferences and are not limited only to that particular embodiment; they may be freely combined with features from other embodiments, where technically feasible, and may form preferred combinations of features. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein. Further, sectional headings are provided for organizational purposes and are not to be considered limiting.

### Disease Characterization Classes and Subclasses

The methods and systems described herein relate to distinguishing a first disease characterization class from a second disease characterization class. Disease characterization classes may be defined according to a diagnosis, such as a cancer diagnosis in an individual. For example, a first disease characterization class may be cancer and a second disease characterization class may be non-cancer. The disease characterization classes may relate characterizations of diseases, e.g. cancer that are more specific than presence or absence of the disease. As non-limiting examples, the disease characterization classes may relate to cancer stages or tissue of origin of a cancer.

Disease characterization subclasses are used to distinguish different etiologies that may be within the first disease characterization class or second disease characterization class. For example, non-cancerous tumor (e.g. advanced adenoma) and healthy may be disease characterization subclasses within a disease characterization class of non-cancer. If the disease characterization classes are both cancer types, such as stage I cancer vs stage II cancer, the disease characterization subclasses may be more specific attributes of the samples in the subclass. Depending on the goals of the method and the disease characterization classes that one is trying to distinguish, the diseased characterization classes and subclasses may be variable.

The methods and systems provided herein rely on training samples that are labeled using a disease characterization subclass label. The disease characterization subclass labels are used to identify which of the disease characterization subclasses the sample belongs to. For example, a sample from a patient with advanced adenoma may be in the disease characterization subclass advanced adenoma and the training data from the sample may be labeled as advanced adenoma. The data from the training samples are labeled to identify the samples for identification of which features distinguish the disease characterization subclasses as described herein. The methods require disease characterization subclasses labels, but may not require disease characterization labels. The disease characterization subclass labels can be grouped into the first and the second disease characterization classes.

### Features

The methods described herein comprise generating a feature group. Features are characteristics of the data that can be used downstream as independent variables in machine learning tasks such as classification or feature selection. Features are associated with values that describe the feature in each of one or more samples (e.g. training samples). Features may be categorical or numerical metrics associated with a subgenomic interval. As used herein the features are associated with genomic, transcriptomic, epigenomic, or microbiome properties. The features are properties of a sample that can be characterized using genomic, transcriptomic, epigenomic, or microbiome techniques. The features may be associated with a plurality of subject intervals, a plurality of genes, a plurality of epigenetic states and/or a plurality of genetic alterations. For example, a feature may be methylation status at a specific subject interval or gene expression for a gene. In some aspects, the subject intervals comprise subgenomic intervals.

The features associated with subject intervals may comprise a chromosome, a genomic starting location, and a genomic ending location. The features may be associated with a plurality of genes or a plurality of transcripts. The features associated with a plurality of genes may comprise a gene name, a subject interval comprising the gene, or a transcript associated with expression of the gene. The features may be associated with transcriptomic regions and may associated with transcript IDs. The features may be a plurality of epigenetic states at a subject interval. The epigenetic states may comprise methylation states, histone modification states, chromatin modification states, or chromatin accessibility states. Chromatin accessibility states may be fragmentomic end points in cfDNA or ctDNA. In some aspects, the subject intervals comprise subgenomic intervals.

The plurality of features may be associated with genomic alterations. Genomic alterations may be single nucleotide variants, insertions, deletions, copy number variations, structural variations or chromosomal alterations. The genomic alterations may comprise known SNP or variant IDs. The genomic alterations may be identified based on genomic locations and modification using methods known in the art. Features associated with genomic alterations may relate to allele frequency and/or variant allele frequency for a specific allele. Allele frequency and/or variant allele frequency may be measured in genomic read data or transcriptomic read data.

In some aspects, the features may be associated with microbiome analysis. The features may be associated with a plurality of microbacterium species, such as but not limited to abundance, variation, and or characteristics of the microbacterium species. The features may relate to integration of viral genomes.

### Methods for Generating a Feature Group

The disclosed methods for generating a feature group for distinguishing a first disease characterization class from a second disease characterization class. The methods comprise receiving training data comprising values for features for a plurality of training samples. The plurality of training samples comprise a disease characterization subclass label, wherein the disease characterization subclass label represents one of a plurality of disease characterization subclasses to which the training sample belongs. The plurality of training samples represent a plurality of disease characterization subclass labels. Pairwise representative features distinguishing the samples from each of the disease characterization subclasses from the samples of the other disease characterization subclasses.

The disease characterizing subclasses are assigned to one of the first disease characterization class or second disease characterization class. A first subset of the plurality of the disease characterization subclass labels as belonging to the first disease characterization class and a second subset of the plurality of the disease characterization subclass labels as belonging to the second disease characterization class.

The pairwise subclass representative features are subset to include the pairwise subclass representative features distinguishing disease characterization subclass between the first and second class and not to include the pairwise subclass representative features distinguishing disease characterization subclasses in a common class.

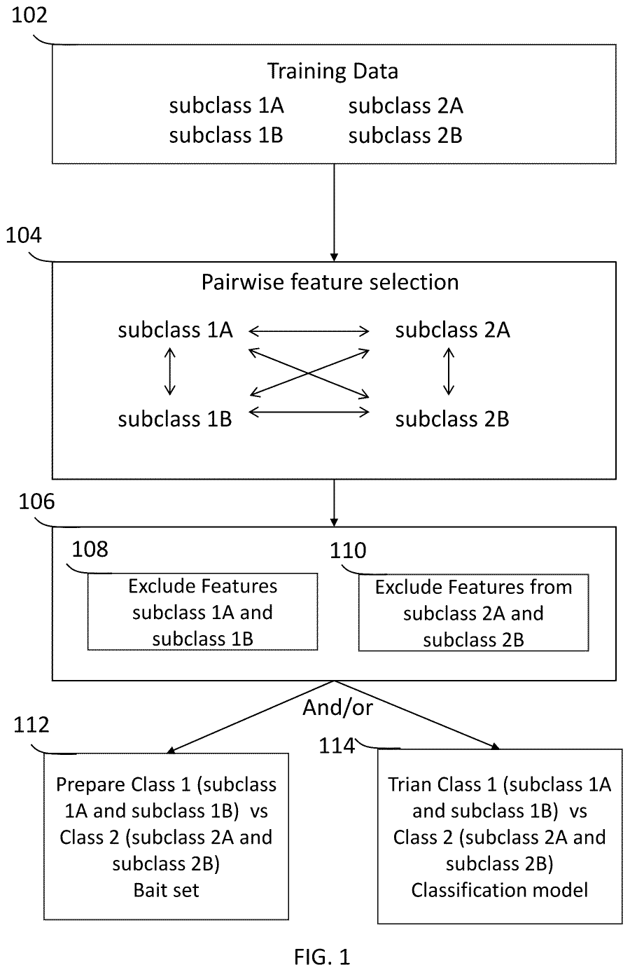

FIG. 1 provides a non-limiting example of methods for generating a feature group for distinguishing a first disease characterization class from a second disease characterization class and applications of the feature group according to methods and systems described herein.

Process 100 can be performed, for example, using one or more electronic devices implementing a software platform. In some examples, process 100 is performed using a client-server system, and the blocks of process 100 are divided up in any manner between the server and a client device. In other examples, the blocks of process 100 are divided up between the server and multiple client devices. Thus, while portions of process 100 are described herein as being performed by particular devices of a client-server system, it will be appreciated that process 100 is not so limited. In other examples, process 100 is performed using only a client device or only multiple client devices. In process 100, some blocks are, optionally, combined, the order of some blocks is, optionally, changed, and some blocks are, optionally, omitted. In some examples, additional steps may be performed in combination with the process 100. Accordingly, the operations as illustrated (and described in greater detail below) are exemplary by nature and, as such, should not be viewed as limiting.

At 102, training data comprising values for features for a plurality of training samples comprising a disease characterization subclass label, wherein the disease characterization subclass label represents one of a plurality of disease characterization subclasses to which the training sample belongs, and wherein the plurality of training samples represents a plurality of disease characterization subclass labels.

In some aspects, the training data comprises genomic, epigenomic, or transcriptomic data. In some aspects the features associated with a plurality of subject intervals, a plurality of genes, plurality of epigenetic states and/or a plurality of genetics alterations. In some aspects, the subject intervals comprise subgenomic intervals.

The training data comprises values associated with the features for each of the training samples. The values may be genomic, epigenomic or transcriptomic values related to the features in the plurality of features. The values may be continuous variables, such as measurements or quantifications. In some aspects the values comprise mRNA expression values or protein expression values. In some aspects, the values comprise methylation measurements such as weighted methylation fraction (WMethfrac) or weighted methylation entropy (WEntropy). WMethfrac may be calculated in each sample for a given genomic interval containing one or more CpG sites as the sum of the number of observed methylation events across all CpGs in the interval is divided the sum of individual CpG coverage values in the interval (total number of observed events, methylated or unmethylated, at each CpG summed). WEntropy may be calculated as the Shannon entropy. In some aspects, in each sample for a given interval with one or more CpG sites as the total number of observed methylation events across all CpGs in the interval is calculated. Similarly, the total number of observed unmethylated CpG events may be calculated. These counts are converted to fractions, the methylation fraction (ex. p) and the unmethylation fraction (ex. q=1−p). The Shannon entropy of the interval can be calculated according to the formula −p*log 2(p)−q*log 2(q). In some aspects, the values may be categorical variables, such as presence or absence of the feature in the sample. In some aspects, the values comprise a genetic alteration status. In some aspects, the training data comprise microbiome data.

The training data may comprise between about 1000 to about 150000 features and associated values for the training samples. The training data may comprise about 1000, about 2000, about 3000, about 4000, about 5000, about 6000, about 7000, about 8000, about 9000, about 10000, about 12000, about 15000, about 20000, about 25000, about 30000, about 35000, about 40000, about 45000, about 50000, about 60000, about 65000, about 70000, about 75000, about 80000, about 85000, about 90000, about 95000, about 100000, about 110000, about 120000, about 130000, about 140000, about 150000 or more than 150000 features and associated values for the training samples. The training data may comprise between about 1000 and about 95000, between about 1000 and about 90000, between about 1000 and about 80000, between about 1000 and about 70000, between about 1000 and about 60000, between about 1000 and about 50000, between about 1000 and about 40000, between about 1000 and about 30000, between about 1000 and about 20000, or between about 1000 and about 10000 features and associated values for the training samples. The training data may comprise between about 10000 and about 150000, between about 20000 and about 150000, between about 30000 and about 150000, between about 40000 and about 150000, between about 50000 and about 150000, between about 60000 and about 150000, between about 70000 and about 150000, between about 80000 and about 150000, between about 90000 and about 150000, between about 100000 and about 150000, between about 110000 and about 150000, between about 120000 and about 150000, between about 130000 and about 150000, or between about 140000 and about 150000 features and associated values for the training samples.

In some aspects, the training samples are collected from subjects who have been diagnosed with a disease associated with one of the subclasses or healthy individuals. Samples from healthy individuals may be labeled “panel of normal” (PoN) or non-cancer (NonCan). In some aspects, the training samples comprise tissue or liquid biopsy samples. In some aspects, the training samples comprise a tissue biopsy, a liquid biopsy, or a normal control. Training data may be collected from training samples by sequencing a plurality of nucleic acid molecules obtained from the training samples.

As in 102, the training data comprises data labeled with subclass 1A, subclass 1B, subclass 2A, and subclass 2B. The subclasses in 102, are exemplary. In some aspects, the disease characterization subclass labels at block 102, may relate to a pathological status, tissue of origin, or stage of a disease. The characterization may be heathy, PON or NonCan. In some aspects, the disease characterization subclasses comprise, colorectal cancer, advanced adenoma, lung cancer, lung adenocarcinoma, lung squamous-cell carcinoma, esophageal cancer, stomach cancer, liver cancer, bile duct cancer, pancreatic cancer, bladder cancer, and ovarian cancer, non-advanced adenoma and non-cancer. In some aspects, the disease characterization subclasses comprise stage I cancer, stage II cancer, stage III cancer, stage IV cancer, stage unknown. In some aspects, the disease characterization subclasses comprise colorectal cancer stage I, colorectal cancer stage II, colorectal cancer stage III, colorectal cancer stage IV, lung cancer stage I, lung cancer stage II, lung cancer stage III, lung cancer stage IV.

At 104, pairwise feature selective is performed. Pairwise subclass representative features that distinguish the disease characterization subclasses from other disease characterization subclasses in the plurality of disease characterization subclasses are selected. As demonstrated using the exemplary subclasses (subclass 1A, subclass 1B, subclass 2A, subclass 2B), pairwise feature selection may comprise selecting features differentiating each of the subclasses. As non-limiting examples, features distinguishing subclass 1A from subclass 1B, subclass 1A from subclass 2A, subclass 1A from subclass 2B, subclass 1B from subclass 2A, subclass 1B from subclass 2B, subclass 2A from subclass 2B.

In some aspects, multiple features may be associated with a single subgenomic interval, for example WMethfrac and WEntropy. In some aspects, pairwise feature selection may be performed separately for the different values associated with the features. Pairwise feature selection may be performed with multiple values associated with the same features together or individually.

Pairwise feature selection at block 104, may comprise use of one or more of an information gain model, Random forest model, ANOVA model, correlation filtering model, T-test model or any other feature selection methods known in the art. In some aspects, selecting pairwise features may comprise applying one or more feature selection model, and preparing a union of top features. In some aspects, two or more feature selection method are performed sequentially.

In some aspects, the pairwise features comprise any significant feature identified during feature selection. In some aspects, pairwise feature selection comprises ranking features based on significance or effect size. In some aspects, the pairwise features comprise the top features in the ranking by significance or effect size. The top features may comprise the top about 99%, about 95%, about 90%, about 85%, about 80%, about 75%, about 70%, about 65%, about 60%, about 55%, o about 50%. The top features may comprise between the top about 50% and top about 99%, between the top about 50% and top about 95%, between the top about 50% and top about 90%, between the top about 50% and top about 85%, between the top about 50% and top about 80%, between the top about 50% and top about 75%, between the top about 50% and top about 70%, between the top about 50% and top about 65%, between the top about 50% and top about 60%, or between the top about 50% and top about 55%. The top features may comprise between the top about 55% and top about 99%, between the top about 60% and top about 99%, between the top about 65% and top about 99%, between the top about 70% and top about 99%, between the top about 75% and top about 99%, between the top about 80% and top about 99%, between the top about 55% and top about 99%, between the top about 90% and top about 99%, or between the top about 95% and top about 99%.

In some aspects, pairwise feature selection comprises generation of an annotation table of top features for each of the binary classifications. In some aspects, the annotation table comprises the comparisons between the classification as columns and the features as rows. The annotation table may comprise 0s and 1s to demonstrate whether a feature was identified as important for distinguishing the subclassifications. Table 1. provides a non-limiting example of an annotation table generated according to the methods described herein. In Table 1, the features are subject intervals and the values are methylation measurements (e.g. WMethfrac, WEntropy).

TABLE 1
Exemplary Annotation Table

| | Wmethfrac_{—} | Wmethfrac_{—} | Wentropy_{—} | Wentropy_{—} | Wmethfrac_{—} | Wentropy_{—} |
|---|---|---|---|---|---|---|
| | PoNNCNAA_{—} | PoN_{—} | PoNNCNAA_{—} | PoN_{—} | PoN_{—} | PoN_{—} |
| regionID | CRC01 | CRC01 | CRC01 | CRC01 | NCNAA | NCNAA |
| chr10_{—} | 1 | 1 | 1 | 1 | 0 | 0 |
| 25463755_{—} | | | | | | |
| 25464090 | | | | | | |
| chr10_{—} | 1 | 1 | 1 | 1 | 1 | 0 |
| 43361552_{—} | | | | | | |
| 43362097 | | | | | | |
| chr14_{—} | 1 | 1 | 1 | 1 | 0 | 1 |
| 29237269_{—} | | | | | | |
| 29237833 | | | | | | |
| chr15_{—} | 1 | 1 | 1 | 1 | 1 | 0 |
| 31775567_{—} | | | | | | |
| 31775644 | | | | | | |
| chr19_{—} | 1 | 1 | 1 | 1 | 0 | 0 |
| 45655911_{—} | | | | | | |
| 45655971 | | | | | | |
| chr1_{—} | 1 | 1 | 1 | 1 | 0 | 0 |
| 21011179_{—} | | | | | | |
| 210112054 | | | | | | |
| chr20_{—} | 1 | 1 | 1 | 1 | 0 | 0 |
| 44686058_{—} | | | | | | |
| 44686589 | | | | | | |
| chr21_{—} | 1 | 1 | 1 | 1 | 0 | 0 |
| 48018542_{—} | | | | | | |
| 48018791 | | | | | | |
| chr2_{—} | 1 | 1 | 1 | 1 | 0 | 0 |
| 38301275_{—} | | | | | | |
| 38302992 | | | | | | |
| chr2_{—} | 1 | 1 | 1 | 1 | 1 | 0 |
| 39187355_{—} | | | | | | |
| 39187760 | | | | | | |
| chr4_{—} | 1 | 1 | 1 | 1 | 0 | 0 |
| 49163547_{—} | | | | | | |
| 49163594 | | | | | | |
| chr4_{—} | 1 | 1 | 1 | 1 | 0 | 0 |
| 58040750_{—} | | | | | | |
| 58047464 | | | | | | |
| chr5_{—} | 1 | 1 | 1 | 1 | 1 | 1 |
| 16179063_{—} | | | | | | |
| 16179953 | | | | | | |
| chr5_{—} | 1 | 1 | 1 | 1 | 0 | 0 |
| 45695661_{—} | | | | | | |
| 45696511 | | | | | | |
| chr6_{—} | 1 | 1 | 1 | 1 | 1 | 0 |
| 105388367_{—} | | | | | | |
| 105388884 | | | | | | |
| chr6_{—} | 1 | 1 | 1 | 1 | 0 | 1 |
| 105388895_{—} | | | | | | |
| 105389318 | | | | | | |
| chr6_{—} | 1 | 1 | 1 | 1 | 1 | 0 |
| 110448819_{—} | | | | | | |
| 11045001 | | | | | | |
| chr7_{—} | 1 | 1 | 1 | 1 | 0 | 0 |
| 8481974_{—} | | | | | | |
| 8482762 | | | | | | |
| chr9_{—} | 1 | 1 | 1 | 1 | 0 | 0 |
| 140773009_{—} | | | | | | |
| 140773320 | | | | | | |

At 106, a feature group is generated. Block 106 may comprise selecting a first subset of the plurality of the disease characterization subclass labels as belonging to the first disease characterization class and a second subset of the plurality of the disease characterization subclass labels as belonging to the second disease characterization class. The first subset and second subset of the disease characterization subclass labels represent the subclasses that are within the first disease characterization class and within the second disease characterization class. The subclass labels in each of the first subset and second subset are flexible and biologically informed. A user may select the subsets based on similarity in pathology and/or the intended disease characterization distinguishing task the method is being used to generate a feature group for. By way of illustration, in FIG. 1, the first subset of the plurality of disease characterization subclass labels belonging to the first disease characterization class comprise subclass 1A and subclass 1B. The second subset of the plurality of disease characterization subclass labels belonging to the second disease characterization class comprise subclass 2A and subclass 2B. The subsets are selected so that the subclasses that represent class 1 (subclass 1A and subclass 1B) are grouped together and the subclasses that represent class 2 (subclass 2A and subclass 2B) are grouped together.

In some aspects, first disease characterization class is cancer and the second disease characterization class is non cancer. The subsets may be selected to include samples collected from individuals with different forms of cancer in the first subset and samples from NonCan pathology or PoN in the second subset. The methods may be used for early detection analysis. In some aspects, the first subset of disease characterization subclasses comprises, colorectal cancer, advanced adenoma, lung cancer, lung adenocarcinoma, lung squamous-cell carcinoma, esophageal cancer, stomach cancer, liver cancer, bile duct cancer, pancreatic cancer, bladder cancer, and ovarian cancer, and the second subset of disease characterization subclasses comprises non-advanced adenoma and non-cancer.

In some aspects, the first disease characterization class and second disease characterization class represent different cancer types, tissues of origin, and/or cancer stage. The methods may be used for early detection, diagnostics, and treatment monitoring. In some aspects, the first disease characterization class is colorectal cancer, and the second disease characterization class is lung cancer. In some aspects, the first subset of disease characterization subclasses comprises, colorectal cancer stage I, colorectal cancer stage II, colorectal cancer stage III, colorectal cancer stage IV, and the second subset of disease characterization subclasses comprises lung cancer stage I, lung cancer stage II, lung cancer stage III, lung cancer stage IV. In some aspects, the first subset of disease characterization subclasses comprise stages of any of the cancers described herein and the second disease characterization subclasses comprise stages of a different cancer described herein.

At 108 and 110, a subset of pairwise subclass representative features are selected, wherein the subset of pairwise subclass representative features distinguish disease characterization subclasses from within a common disease characterization class. The features may be selected and excluded from the pairwise subclass representative features identified at 104. By way of illustration, at block 108, the pairwise representative features identified in the comparison between subclass 1A and subclass 1B are selected and excluded. These features represent features identified as distinguishing subclasses in a common disease characterization class meaning the subclasses were selected in the same subset. Here, subclass 1A and subclass 1B are both in the first subset representing the first disease characterization class. Similarly, at 110, by way of illustration, the pairwise representative features identified in the comparison between subclass 2A and subclass 2B are selected and excluded. Here, subclass 2A and subclass 2B are both in the second subset representing the second disease characterization class. As described herein, selecting and excluding the features identified in a common class remove features that would otherwise include within disease characterization class variation in downstream analysis. The feature group remaining after exclusion at 108 and 110 may represent the feature group for distinguishing the first disease characterization class (e.g. Class 1) from the second disease characterization class (e.g. class 2).

In some aspects, an annotation table as shown in Table 1, may be used for selecting and excluding features. As shown in Table 1, features identified as important for a comparison are labeled with a 1. In some aspects, features with a 1 designation for only for within disease characterization classes may be removed.

In some aspects, features significant for additional comparisons of subclassifications may be performed using the feature group after removal of the features as shown in 108 and 110. In some aspects, the methods may comprise selecting features unique to a single subclassification. In some aspects, the methods may comprise identifying features according to a 1:Rest scheme. As an example, a 1:Rest scheme may include selecting features significant for the comparison subclass 1A vs. subclass 1B, subclass 2A, and subclass 2B. The 1:Rest features may be selected from all the features in the training set or from the previously identified feature group. Features identified in a 1:Rest scheme may be informative for subgroup specific variation.

In some aspects, the methods comprise generating the training data. The training data may be generated using nucleic acid extraction and processing methods disclosed herein. The training data may be generated from samples as described herein. The training data may be generated using microbiome profiling and analysis method known in the art and described herein.

### Methods for Designing a Plurality of Nucleic Acid Baits

Provided herein are methods for designing and/or making a plurality of nucleic acid baits. The methods for designing a plurality of nucleic acid baits comprise generating a feature group according to the methods described herein and designing a plurality of nucleic acid baits corresponding to genomic regions covering on or more features of the feature group. The methods for making a plurality of nucleic acid baits comprise generating a feature group according to the methods described herein and making the plurality of nucleic acid baits corresponding to genomic regions covering one or more features in the feature group. The nucleic acid baits may be used for targeted sequencing of nucleic acids in samples. The target sequencing may improve classification of the sample as belonging to the first disease characterization class or second disease characterization class. Using targeted sequencing with the nucleic acid baits may increase sequence coverage for informative genomic regions thus reducing wasted sequencing reads and/or requiring less sequencing resources.

According to the exemplary embodiment of FIG. 1, at block 112, the feature group selected at block 106 is used to prepare a class 1 vs class 2 bait set. The bait set may comprise nucleic acid baits covering one or more of the features selected at block 112.

### Nucleic Acid Baits

In some embodiments, the nucleic acid bait in the plurality of nucleic acid baits comprises a capture nucleic acid molecule configured to hybridize to a target nucleic acid molecule comprising a nucleic acid molecule within a genomic region of a selected feature or a fragment or portion thereof.

In some embodiments, the capture nucleic acid molecule is configured to hybridize to a fragment of the nucleic acid molecule within a genomic region of a selected feature. In some embodiments, the fragment comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the capture nucleic acid molecule is between about 5 and about 25 nucleotides, between about 5 and about 300 nucleotides, between about 100 and about 300 nucleotides, between about 130 and about 230 nucleotides, or between about 150 and about 200 nucleotides. In some embodiments, the fragment comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the capture nucleic acid molecule comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length.

In some embodiments, the capture nucleic acid molecule is a DNA, RNA, or a DNA/RNA molecule. In some embodiments, the capture nucleic acid molecule comprises any of between about 50 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the capture nucleic acid molecule comprises any of between about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the capture nucleic acid molecule comprises about 150 nucleotides. In some embodiments, the capture nucleic acid molecule is about 150 nucleotides. In some embodiments, the capture nucleic acid molecule comprises about 170 nucleotides. In some embodiments, the capture nucleic acid molecule is about 170 nucleotides.

In some embodiments, a nucleic acid bait provided herein comprises a DNA, RNA, or a DNA/RNA molecule. In some embodiments, a nucleic acid bait provided herein includes a label or a tag. In some embodiments, the label or tag is a radiolabel, a fluorescent label, an enzymatic label, a sequence tag, biotin, or another ligand. In some embodiments, a nucleic acid bait provided herein includes a detection reagent such as a fluorescent marker. In some embodiments, a nucleic acid bait provided herein includes (e.g., is conjugated to) an affinity tag, e.g., that allows capture and isolation of a hybrid formed by a nucleic acid bait and a nucleic acid hybridized to the nucleic acid bait. In some embodiments, the affinity tag is an antibody, an antibody fragment, biotin, or any other suitable affinity tag or reagent known in the art. In some embodiments, a nucleic acid bait is suitable for solution phase hybridization.

Nucleic acid baits can be produced (e.g. made or manufactured) and used according to methods known in the art, e.g., as described in WO2012092426A1 and/or or in Frampton et al (2013) Nat Biotechnol, 31:1023-1031, incorporated herein by reference. For example, biotinylated baits (e.g., RNA baits) can be produced by obtaining a pool of synthetic long oligonucleotides, originally synthesized on a microarray, and amplifying the oligonucleotides to produce the bait sequences. In some embodiments, the baits are produced by adding an RNA polymerase promoter sequence at one end of the bait sequences, and synthesizing RNA sequences using RNA polymerase. In one embodiment, libraries of synthetic oligodeoxynucleotides can be obtained from commercial suppliers, such as Agilent Technologies, Inc., and amplified using known nucleic acid amplification methods.

In some embodiments, a nucleic acid bait provided herein is between about 100 nucleotides and about 300 nucleotides. In some embodiments, a nucleic acid bait provided herein is between about 130 nucleotides and about 230 nucleotides. In some embodiments, a nucleic acid bait provided herein is between about 150 nucleotides and about 200 nucleotides. In some embodiments, a nucleic acid bait provided herein comprises a target-specific bait sequence (e.g., a capture nucleic acid molecule described herein) and universal tails on each end. In some embodiments, the target-specific sequence, e.g., a capture nucleic acid molecule described herein, is between about 40 nucleotides and about 300 nucleotides. In some embodiments, the target-specific sequence, e.g., a capture nucleic acid molecule described herein, is between about 100 nucleotides and about 200 nucleotides. In some embodiments, the target-specific sequence, e.g., a capture nucleic acid molecule described herein, is between about 120 nucleotides and about 170 nucleotides. In some embodiments, the target-specific sequence, e.g., a capture nucleic acid molecule described herein, is about 150 nucleotides or about 170 nucleotides. In some embodiments, a nucleic acid bait provided herein comprises an oligonucleotide comprising about 200 nucleotides, of which about 150 nucleotides or about 170 nucleotides are target-specific (e.g., a capture nucleic acid molecule described herein), and the other 50 nucleotides or 30 nucleotides (e.g., 25 or 15 nucleotides on each end of the nucleic acid bait) are universal arbitrary tails, e.g., suitable for PCR amplification.

The nucleic acid baits described herein can be used for selection of exons and short target sequences.

In some embodiments, a nucleic acid bait of the disclosure distinguishes a nucleic acid, e.g., a genomic or transcribed nucleic acid, e.g., a cDNA or RNA, from a genomic region in a selected feature described herein.

In some aspects, two or more nucleic acid baits are designed for a single feature. The number of nuclei acid baits designed to cover a feature (e.g. genomic region) may depend on the size of the feature (e.g. number of bases in the genomic region). The number of nucleic acid baits may also depend on the complexity of the genomic region represented by the feature.

### Methods for Training a Classification Model and for Classifying a Test Sample

Provided herein are methods for training a machine learning model (e.g. classification model) to classify a test sample as a first disease characterization class or a second disease characterization class. The method comprise receiving, at one or more processors, training data comprising values for features for a plurality of training samples, comprising a disease characterization subclass label, wherein the disease characterization subclass label represents one of a plurality of disease characterization subclasses the training sample belongs to and wherein the plurality of training samples represents a plurality of disease characterization subclass labels, and wherein the features comprise a plurality of features from a feature group generated according to the methods described herein; selecting, by one or more processors, a first subset of the plurality of the disease characterization subclass labels as belonging to the first disease characterization class and a second subset of the plurality of the disease characterization subclass labels as belonging to the second disease characterization class; training, by the one or more processors a machine learning model to classify a test sample as the first disease characterization class or the second disease characterization class using the training data, and the plurality of features from the feature group. The machine learning model may be a classification model as described herein. In some aspects, the methods comprise generating the training data as described herein. In some aspects, the training data are the same training data used for selecting the features according to the methods described herein. In some aspects, new training data may be used for training the model to classify a test sample as belonging to the first disease class or the second disease class.

According to the exemplary embodiment of FIG. 1, at block 114, the feature group selected at block 106 is used to train a classification model for distinguishing a sample as belonging to class 1 or class 2.

In some aspect, the machine learning model is trained using one or more of the plurality of features from the feature group generated to distinguish a first disease characterization class from a second characterization class according to the methods described herein. In some aspects, methods comprise performing a dimensional rejection method on the feature group before training the machine learning model. The dimensionality reduction methods may comprise principal component analysis (PCA) or UMAP. In some aspects, the machine learning model is trained with features from the feature group before and/or after dimensionality reduction.

In some aspects, the machine learning model is a classification machine learning model. The classification machine learning model may be any classification model known in the art. In some aspects, the machine learning model comprises one or more of a GLMnet, Random Forest, Support Vector Machine, Naïve Boost, Naïve Bayesian, XGboost, or Logistic regression model.

In some instances, training the machine learning model comprises selecting the hyperparameters and/or model architecture with the best performance. The best performance may be calculated by assessing the ability of the trained machine learning model to correctly classify previously unseen (potentially holdout samples from the training dataset) as the first disease characterization class or second disease characterization class. In some aspects, iterative resampling and model training methods known in the art may be used.

Any of a variety of machine learning approaches & algorithms (where a machine learning model, as referred to herein, comprises a trained machine learning algorithm) may be used in implementing the disclosed methods. For example, the machine learning model may comprise a supervised learning model (i.e., a model trained using labeled sets of training data), an unsupervised learning model (i.e., a model trained using unlabeled sets of training data), a semi-supervised learning model (i.e., a model trained using a combination of labeled and unlabeled training data), a self-supervised learning model, or any combination thereof. In some examples, the machine learning model can comprise a deep learning model (i.e., a model comprising many layers of coupled “nodes” that may be trained in a supervised, unsupervised, or semi-supervised manner).

In some instances, one or more machine learning models (e.g., 1, 2, 3, 4, 5, 6, 7, 8, 9, 10, or more than 10 machine learning models), or a combination thereof, may be utilized to implement the disclosed methods.

In some instances, the one or more machine learning models may comprise statistical methods for analyzing data. The machine learning models may be used for classification and/or regression of data. The machine learning models can include, for example, neural networks, support vector machines, decision trees, ensemble learning (e.g., bagging-based learning, such as random forest, and/or boosting-based learning), k-nearest neighbors algorithms, linear regression-based models, and/or logistic regression-based models. The machine learning models can comprise regularization, such as L1 regularization and/or L2 regularization. The machine learning models can include the use of dimensionality reduction techniques (e.g., principal component analysis, matrix factorization techniques, and/or autoencoders) and/or clustering techniques (e.g., hierarchical clustering, k-means clustering, distribution-based clustering, such as Gaussian mixture models, or density-based clustering, such as DBSCAN or OPTICS). The one or more machine learning models can comprise solving, e.g., optimizing, an objective function over multiple iterations based on a training data set. The iterative solving approach can be used even when the machine learning model comprises a model for which there exists a closed-form solution (e.g., linear regression).

In some instances, the machine learning models can comprise artificial neural networks (ANNs), e.g., deep learning models. For example, the one or more machine learning models/algorithms used for implementing the disclosed methods may include an ANN which can comprise any of a variety of computational motifs/architectures known to those of skill in the art, including, but not limited to, feedforward connections (e.g., skip connections), recurrent connections, fully connected layers, convolutional layers, and/or pooling functions (e.g., attention, including self-attention). The artificial neural networks can comprise differentiable non-linear functions trained by backpropagation.

Artificial neural networks, e.g., deep learning models, generally comprise an interconnected group of nodes organized into multiple layers of nodes. For example, the ANN architecture may comprise at least an input layer, one or more hidden layers (i.e., intermediate layers), and an output layer. The ANN or deep learning model may comprise any total number of layers (e.g., 3, 4, 5, 6, 7, 8, 9, 10, 15, 20, or more than 20 layers in total), and any number of hidden layers (e.g., 1, 2, 3, 4, 5, 6, 7, 8, 9, 10, 15, 20, or more than 20 hidden layers), where the hidden layers function as trainable feature extractors that allow mapping of a set of input data to a preferred output value or set of output values. Each layer of the neural network comprises a plurality of nodes (e.g., at least 10, 25, 50, 75 100, 200, 300, 400, 500, 600, 700, 800, 900, 1000, 2000, 3000, 4000, 5000, 6000, 7000, 8000, 9000, 10,000, or more than 10,000 nodes). A node receives input data (e.g., genomic feature data (such as variant sequence data, methylation status data, etc.), non-genomic feature data (e.g., digital pathology image feature data), or other types of input data (e.g., patient-specific clinical data)) that comes either directly from one or more input data nodes or from the output of one or more nodes in previous layers, and performs a specific operation, e.g., a summation operation. In some cases, a connection from an input to a node is associated with a weight (or weighting factor). In some cases, the node may, for example, sum up the products of all pairs of inputs, Xᵢ, and their associated weights, Wᵢ. In some cases, the weighted sum is offset with a bias, b. In some cases, the output of a node may be gated using a threshold or activation function, f, where f may be a linear or non-linear function. The activation function may be, for example, a rectified linear unit (ReLU) activation function or other function such as a saturating hyperbolic tangent, identity, binary step, logistic, arcTan, softsign, parameteric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network (or other machine learning architecture), can be “taught” or “learned” in a training phase using one or more sets of training data (e.g., 1, 2, 3, 4, 5, or more than 5 sets of training data) and a specified training approach configured to solve, e.g., minimize, a loss function. For example, the adjustable parameters for an ANN (e.g., deep learning model) may be determined based on input data from a training data set using an iterative solver (such as a gradient-based method, e.g., backpropagation), so that the output value(s) that the ANN computes (e.g., a classification of a sample or a prediction of a disease outcome) are consistent with the examples included in the training data set. The training of the model (i.e., determination of the adjustable parameters of the model using an iterative solver) may or may not be performed using the same hardware as that used for deployment of the trained model.

In some instances, the disclosed methods may comprise retraining any of the machine learning models (e.g., iteratively retraining a previously trained model using one or more training data sets that differ from those used to train the model initially). In some instances, retraining the machine learning model may comprise using a continuous, e.g., online, machine learning model, i.e., where the model is periodically or continuously updated or retrained based on new training data. The new training data may be provided by, e.g., a single deployed local operational system, a plurality of deployed local operational systems, or a plurality of deployed, geographically-distributed operational systems. In some instances, the disclosed methods may employ, for example, pre-trained ANNs, and the pre-trained ANNs can be fine-tuned according to an additional dataset that is inputted into the pre-trained ANN.

Provided herein are methods for classifying a test sample for a subject as a first disease characterization class or a second disease characterization class. The methods comprise, receiving, at one or more processors, test data from a test sample collected from the subject, test data comprising values for a plurality of features of a feature group generated according to the method of any of the methods described herein, and classifying the test samples as the first disease characterization class or the second disease characterization class based on the output from the classification model. In some aspects, the subject is suspected of having or is determined to have cancer, such as any of the cancers described herein. In some aspects, the subject is not suspected of having cancer or is not at risk of having cancer. The model may comprise a classification model trained according to the methods described herein. The methods may comprise the model trained according to the example embodiment in FIG. 1, at block 114.

The test sample may comprise a tissue biopsy sample, a liquid biopsy sample, or a normal control as described herein. The methods may comprise obtaining the test sample from the subject, isolating nucleic acids and performing a sequencing method as described herein.

### Methods of Use

In some instances, the disclosed methods may further comprise one or more of the steps of: (i) obtaining the sample from the subject (e.g., a subject suspected of having or determined to have cancer), (ii) extracting nucleic acid molecules (e.g., a mixture of tumor nucleic acid molecules and non-tumor nucleic acid molecules) from the sample, (iii) ligating one or more adapters to the nucleic acid molecules extracted from the sample (e.g., one or more amplification primers, flow cell adaptor sequences, substrate adapter sequences, or sample index sequences), (iv) performing a methylation conversion reaction to convert, e.g., non-methylated cytosine to uracil, (v) amplifying the nucleic acid molecules (e.g., using a polymerase chain reaction (PCR) amplification technique, a non-PCR amplification technique, or an isothermal amplification technique), (vi) capturing nucleic acid molecules from the amplified nucleic acid molecules (e.g., by hybridization to one or more bait molecules, where the bait molecules each comprise one or more nucleic acid molecules that each comprising a region that is complementary to a region of a captured nucleic acid molecule), (vii) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, (viii) combining the nucleic acid sequence data (including, e.g., variant data, copy number data, methylation status data, etc., of the sequenced nucleic acid molecules) with other biomarker data modalities including, but not limited to, proteomics-based biomarker data (e.g., the detection of specific polypeptides, such as proteins) or fragmentomics-based biomarker data (e.g., the detection of certain attributes related to nucleic acid fragments, such as fragment size or the sequences of fragment ends), to determine, for example, the presence of ctDNA in the sample and/or to determine a diagnostic, prognostic, and/or treatment response prediction for the subject, and (ix) generating, displaying, transmitting, and/or delivering a report (e.g., an electronic, web-based, or paper report) to the subject (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in the graphical user interface of an online or web-based healthcare portal. In some instances, the report is transmitted via a computer network or peer-to-peer connection.

The disclosed methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA). In some instances, the cell-free DNA (cfDNA), or a portion thereof, may comprise circulating tumor DNA (ctDNA). In some instances, the liquid biopsy sample may comprise a combination of cell-free DNA (cfDNA) and circulating tumor DNA (ctDNA).

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods for training and using a machine learning model to classify a test samples as a first disease characterization class or a second disease characterization class may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods for training and using a machine learning model to classify a test sample as a first disease characterization class or a second disease characterization class may be used to select a subject (e.g., a patient) for a clinical trial based on a classification of the test sample as a first disease characterization class or a second disease characterization class. In some instances, patient selection for clinical trials based on, e.g., identification of features in the identified feature group may accelerate the development of targeted therapies and improve the healthcare outcomes for treatment decisions.

In some instances, the disclosed methods classifying a test sample as a first disease characterization class or a second disease characterization class may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy, an immunotherapy, a neoantigen-based therapy, surgery, or any combination thereof.

In some instances, the anti-cancer therapy or treatment may comprise a targeted anti-cancer therapy or treatment (e.g., a monoclonal antibody-based therapy, an enzyme inhibitor-based therapy, an antibody-drug conjugate therapy, a hormone therapy, and/or a targeted radiotherapy) that targets specific molecules required for cancer cell growth, division, and spreading. In some instances, the targeted anti-cancer therapy or treatment may comprise abemaciclib (Verzenio), abiraterone acetate (Zytiga), acalabrutinib (Calquence), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

In some instances, the anti-cancer therapy or treatment may comprise an immunotherapy (e.g., a cancer treatment that acts by stimulating the immune system to fight cancer). In some instances, the immunotherapy can be, for example, an immune system modulator (e.g., a cytokine, such as an interferon or interleukin), an immune checkpoint inhibitor (such as an anti-PD-1 or anti-PD-L1 antibody), a T-cell transfer therapy (e.g., a tumor infiltrating lymphocyte (TIL) therapy in lymphocytes extracted from a patient's tumor are selected for their ability to recognize tumor cells and propagated prior to reintroduction into the patient, or a CAR T-cell therapy in which a patient's T-cells are modified to express the CAR protein prior to reintroduction into the patient), a monoclonal antibody-based therapy (e.g., a monoclonal antibody that binds to cell surface markers on cancer cells to facilitate recognition by the immune system), or a cancer treatment vaccine (e.g., a vaccine based on tumor cells, tumor-associated neoantigens, or dendritic cells, etc., that stimulates the immune system to fight cancer).

In some instances, the anti-cancer therapy or treatment may comprise a neoantigen-based therapy. Non-limiting examples of neoantigen-based therapies include T-cell receptor (TCR) engineered T-cell (TCR-T) therapies, chimeric antigen receptor T-cell (CAR-T) therapies, TCR bispecific antibody therapies, and cancer vaccines. TCR-T therapies are produced by genetically engineering a patient's T-cells to express T-cell receptors that are specific to neoantigens of interest, and then infusing them back into the patient. CAR-T therapies are produced by genetically engineering a patient's T-cells to express chimeric antigen receptor molecules which contain an intracellular signaling and co-signaling domain as well as an extracellular antigen-binding domain; CAR-T therapies don't always rely on neoantigen presentation, but can be designed to be directed towards neoantigens. TCR bispecific antibody therapies are small, engineered antibody molecules that comprise a neoantigen-specific TCR on one end and a CD3-directed single-chain variable fragment on the other end. Cancer vaccines can include RNA molecules, DNA molecules, peptides, or a combination thereof that are designed to boost the immune system's ability to find and destroy neoantigen-presenting cells.

In some instances, the disclosed methods for classifying a test sample as a first disease characterization class or a second disease characterization class may be used in treating a disease (e.g., a cancer) in a subject. For example, in response to determining whether a sample is classified as a first disease classification (e.g. cancer) using any of the methods disclosed herein, an effective amount of an anti-cancer therapy or anti-cancer treatment may be administered to the subject.

In some instances, the disclosed methods for classifying as a first disease characterization class or a second disease characterization class may be used for monitoring disease progression or recurrence (e.g., cancer or tumor progression or recurrence) in a subject. For example, in some instances, the methods may be used to determine a first classification of a test samples as a first disease characterization class or a second disease characterization class in a first sample obtained from the subject at a first time point, and used to determine a second classification in a second sample obtained from the subject at a second time point, where comparison of the first determination of first classification and the second determination of second classification allows one to monitor disease progression or recurrence. In some instances, the first time point is chosen before the subject has been administered a therapy or treatment, and the second time point is chosen after the subject has been administered the therapy or treatment.

In some instances, the disclosed methods may be used for adjusting a therapy or treatment (e.g., an anti-cancer treatment or anti-cancer therapy) for a subject, e.g., by adjusting a treatment dose and/or selecting a different treatment in response to a change in the determination of the classification of a test sample as a first disease characterization class or a second disease characterization class.

In some instances, classification of a test sample as a first disease characterization class or a second disease characterization class determined using the disclosed methods may be used as a prognostic or diagnostic indicator associated with the sample. For example, in some instances, the prognostic or diagnostic indicator may comprise an indicator of the presence of a disease (e.g., cancer) in the sample, an indicator of the probability that a disease (e.g., cancer) is present in the sample, an indicator of the probability that the subject from which the sample was derived will develop a disease (e.g., cancer) (i.e., a risk factor), or an indicator of the likelihood that the subject from which the sample was derived will respond to a particular therapy or treatment.

In some instances, the disclosed methods for classifying a test sample as a first disease characterization class or a second disease characterization class may be implemented as part of a genomic profiling process that comprises identification of the presence of variant sequences at one or more gene loci in a sample derived from a subject as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), which is a next-generation sequencing (NGS) approach used to assess hundreds of genes (including relevant cancer biomarkers) in a single assay. Inclusion of the disclosed methods for classifying a test sample as a first disease characterization class or a second disease characterization class as part of a genomic profiling process (or inclusion of the output from the disclosed methods for classifying a test sample as a first disease characterization class or a second disease characterization class as part of the genomic profile of the subject) can improve the validity of, e.g., disease detection calls and treatment decisions, made on the basis of the genomic profile by, for example, independently confirming the presence of a first disease classification or second disease classification in a given patient sample.

In some instances, a genomic profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a genomic profile for the subject may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

In some instances, the method can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). The samples may be samples used to generate training data or samples used for generating test data as described herein. Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavage or bronchoalveolar lavage), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly (A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly(A) sitail of mature mRNAs by priming with oligo (dT)-containing oligonucleotides. Methods for depletion, poly (A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at less than 5% tumor cell nuclei. In some instances, the sample may comprise a tumor content of at least 0.1%, at least 1%, at least 2%, at least 3%, at least 4%, at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

In some instances, the sample is a sample suitable for microbiome analysis as described herein. Samples include but are not limited to stool samples, skin swabs, oral swabs, vaginal swabs, and/or blood samples. The samples may comprise microbial communities (e.g. a plurality of microbacterium species) relevant to the disease characterization states described herein, such as a cancer. The samples may comprise microbial DNA that can be used to characterize an abundance of microbacterium species. The sample may comprise microbes from a plurality of taxa. The plurality of taxa may include but are not limited to Abiotrophia, Abiotrophia defectiva, Acidobacteria, Acidovorax, Acinetobacter, Acetanaerobacteria, Actinobacteria, Actinomycetes, Aeromonas, Agrobacterium, Akkermansia, Alistipes, Allobaculum, Aquabacterium, Azonexus, Bacillaceae_1, Bacteroides, Bacteroidetes, Bifidobacterium, Bifidobacterium bifidum, Bryantella, Catonella, Carnobacteriaceae_1, Chryseobacterium, Chryseomonas, Cloacibacterium, Clostridiales, Clostridium, Clostridium difficile, Clostridium tetani, Coriobacterineae, Corynebacteria, Comamonas, Cyanobacteria, Dechloromonas, Delftia, Enterobacter, Enterobacteriaceae, Enterococcus faecalis, Escherichia coli, Erwinia, Exiguobacterium, Firmicutes, Flavimonas, Fusobacteria, Gp1, Gp2, Haemophilus influenza, Helicobacter, Hoidemania, Klebsiella, Klebsiella bacterium, Lachnospiraceae incertae sedis, Lactobacillus, Lactococcus, Leuconostoc, Methylobacterium, Micrococcineae, Mycobacteria, Neisseria, Neisseria meningitides, Novosphingobium, Oligotropha, Pantoea, Paiudibacter, Proteobacteria, Proteus, Pseudomonas, Pseudomonas aeruginosa, Pseudoxanthomonas, Raistonia, Rikeneia, Roseburia, Rubrobacterineae, Serratia, Shinella, Sphingobium, Spirochetes, Sporobacter, Staphylococcus, Staphylococcus aureus, Staphylococcus epidermidis, Staphylococcus mitis, Stenotrophomonas, Streptococcus mutans, Streptococcus pneumoniae, Streptococcus pyogenes, Streptococcus salivarius, Stenotrophomonas, Succinivibrio, Sutterella, Syntrophococcus, Turicibacter, Variovorax, Verrucomicrobia, and Weissella.

In some instances, the sample is collected using a colonoscopy, a fecal occult blood test, a fecal immunochemical test, a fecal-based DNA test, or a blood-based DNA test. In some aspects, the sample is frozen and/or lyophilized.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g., a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypereosinophilia, chronic eosinophilic leukemia, neuroendocrine cancers, carcinoid tumors, and the like.

In some instances, the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR and MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), a gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia. \

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Library Preparation

In some instances, the nucleic acids isolated from the sample may be used to construct a library (e.g., a nucleic acid library as described herein). In some instances, the nucleic acids are fragmented using any of the methods described above, optionally subjected to repair of chain end damage, and optionally ligated to synthetic adapters, primers, and/or barcodes (e.g., amplification primers, sequencing adapters, flow cell adapters, substrate adapters, sample barcodes or indexes, and/or unique molecular identifier sequences), size-selected (e.g., by preparative gel electrophoresis), and/or amplified (e.g., using PCR, a non-PCR amplification technique, or an isothermal amplification technique). In some instances, the fragmented and adapter-ligated group of nucleic acids is used without explicit size selection or amplification prior to hybridization-based selection of target sequences. In some instances, the nucleic acid is amplified by any of a variety of specific or non-specific nucleic acid amplification methods known to those of skill in the art. In some instances, the nucleic acids are amplified, e.g., by a whole-genome amplification method such as random-primed strand-displacement amplification. Examples of nucleic acid library preparation techniques for next-generation sequencing are described in, e.g., van Dijk, et al. (2014), Exp. Cell Research 322:12-20, and Illumina's genomic DNA sample preparation kit.

In some instances, the resulting nucleic acid library may contain all or substantially all of the complexity of the genome. The term “substantially all” in this context refers to the possibility that there can in practice be some unwanted loss of genome complexity during the initial steps of the procedure. The methods described herein also are useful in cases where the nucleic acid library comprises a portion of the genome, e.g., where the complexity of the genome is reduced by design. In some instances, any selected portion of the genome can be used with a method described herein. For example, in certain embodiments, the entire exome or a subset thereof is isolated. In some instances, the library may include at least 95%, 90%, 80%, 70%, 60%, 50%, 40%, 30%, 20%, 10%, or 5% of the genomic DNA. In some instances, the library may consist of cDNA copies of genomic DNA that includes copies of at least 95%, 90%, 80%, 70%, 60%, 50%, 40%, 30%, 20%, 10%, or 5% of the genomic DNA. In certain instances, the amount of nucleic acid used to generate the nucleic acid library may be less than 5 micrograms, less than 1 microgram, less than 500 ng, less than 200 ng, less than 100 ng, less than 50 ng, less than 10 ng, less than 5 ng, or less than 1 ng.

In some instances, a library (e.g., a nucleic acid library) includes a collection of nucleic acid molecules. As described herein, the nucleic acid molecules of the library can include a target nucleic acid molecule (e.g., a tumor nucleic acid molecule, a reference nucleic acid molecule and/or a control nucleic acid molecule; also referred to herein as a first, second and/or third nucleic acid molecule, respectively). The nucleic acid molecules of the library can be from a single subject or individual. In some instances, a library can comprise nucleic acid molecules derived from more than one subject (e.g., 2, 3, 4, 5, 6, 7, 8, 9, 10, 20, 30 or more subjects). For example, two or more libraries from different subjects can be combined to form a library having nucleic acid molecules from more than one subject (where the nucleic acid molecules derived from each subject are optionally ligated to a unique sample barcode corresponding to a specific subject). In some instances, the subject is a human having, or at risk of having, a cancer or tumor.

In some instances, the library (or a portion thereof) may comprise one or more subject intervals. In some aspects, the subject intervals comprise subgenomic intervals. In some instances, a subgenomic interval can be a single nucleotide position, e.g., a nucleotide position for which a variant at the position is associated (positively or negatively) with a tumor phenotype. In some instances, a subgenomic interval comprises more than one nucleotide position. Such instances include sequences of at least 2, 5, 10, 50, 100, 150, 250, or more than 250 nucleotide positions in length. Subgenomic intervals can comprise, e.g., one or more entire genes (or portions thereof), one or more exons or coding sequences (or portions thereof), one or more introns (or portion thereof), one or more microsatellite region (or portions thereof), or any combination thereof. A subgenomic interval can comprise all or a part of a fragment of a naturally occurring nucleic acid molecule, e.g., a genomic DNA molecule. For example, a subgenomic interval can correspond to a fragment of genomic DNA which is subjected to a sequencing reaction. In some instances, a subgenomic interval is a continuous sequence from a genomic source. In some instances, a subgenomic interval includes sequences that are not contiguous in the genome, e.g., subgenomic intervals in cDNA can include exon-exon junctions formed as a result of splicing. In some instances, the subgenomic interval comprises a tumor nucleic acid molecule. In some instances, the subgenomic interval comprises a non-tumor nucleic acid molecule.

### Targeting Gene Loci for Analysis

The methods described herein can be used in combination with, or as part of, a method for evaluating a plurality or set of subject intervals (e.g., target sequences), e.g., from a set of genomic loci (e.g., gene loci or fragments thereof), as described herein.

In some instances, the set of genomic loci evaluated by the disclosed methods comprises a plurality of, e.g., genes, which in mutant form, are associated with an effect on cell division, growth or survival, or are associated with a cancer, e.g., a cancer described herein.

In some instances, the set of gene loci evaluated by the disclosed methods comprises at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, or more than 100 gene loci.

In some instances, the selected gene loci (also referred to herein as target gene loci or target sequences), or fragments thereof, may include subject intervals comprising non-coding sequences, coding sequences, intragenic regions, or intergenic regions of the subject genome. For example, the subject intervals can include a non-coding sequence or fragment thereof (e.g., a promoter sequence, enhancer sequence, 5′ untranslated region (5′ UTR), 3′ untranslated region (3′ UTR), or a fragment thereof), a coding sequence of fragment thereof, an exon sequence or fragment thereof, an intron sequence or a fragment thereof.

In some instances, the gene panel may comprise at least 1, at least 2, at least 3, at least 4, at least 5, at least 6, at least 7, at least 8, at least 9, at least 10, at least 20, at least 30, at least 40, or more than 40 genes.

In some instances, the disclosed methods may be used to generating a feature group by assessing a plurality of sequence reads overlapping in at least 1, at least 2, at least 3, at least 4, at least 5, at least 6, at least 7, at least 8, at least 9, at least 10, at least 20, at least 30, at least 40, or more than 40 gene loci. In some instances, the disclosed methods may be used to generating a feature group by assessing a plurality of sequence reads overlapping in at least 100, at least 200, at least 300, at least 400, at least 500, at least 600, at least 700, at least 800, at least 9000, at least 10000, at least 20000, at least 30000, at least 40000, or more than 40000 gene loci. In some instances, the disclosed methods may be used to generating a feature group by assessing a plurality of sequence reads overlapping in at least 10000, at least 20000, at least 30000, at least 40000, at least 50000, at least 60000, at least 70000, at least 80000, at least 900000, at least 100000, at least 200000, at least 300000, at least 400000, or more than 400000 gene loci.

In some instances, the disclosed methods may be used to generating a feature group by assessing a plurality of sequence reads overlapping in at one or more gene loci. In some aspects, the one or more gene loci comprise between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

In some aspects, the one or more gene loci comprise between 1000 and 2000 loci, between 1000 and 4000 loci, between 1000 and 6000 loci, between 1000 and 8000 loci, between 1000 and 10000 loci, between 10000 and 15000 loci, between 1000 and 20000 loci, between 1000 and 25000 loci, between 1000 and 30000 loci, between 1000 and 35000 loci, between 1000 and 40000 loci, between 1000 and 45000 loci, between 1000 and 50000 loci, between 2000 and 4000 loci, between 2000 and 6000 loci, between 2000 and 8000 loci, between 2000 and 10000 loci, between 2000 and 15000 loci, between 2000 and 20000 loci, between 2000 and 25000 loci, between 2000 and 30000 loci, between 2000 and 35000 loci, between 2000 and 40000 loci, between 2000 and 50000 loci, between 4000 and 6000 loci, between 4000 and 8000 loci, between 4000 and 10000 loci, between 4000 and 15000 loci, between 4000 and 20000 loci, between 4000 and 25000 loci, between 4000 and 3000 loci, between 4000 and 35000 loci, between 4000 and 40000 loci, between 4000 and 50000 loci, between 6000 and 8000 loci, between 6000 and 10000 loci, between 6000 and 15000 loci, between 6000 and 20000 loci, between 6000 and 25000 loci, between 6000 and 30000 loci, between 6000 and 35000 loci, between 6000 and 40000 loci, between 6000 and 50000 loci, between 8000 and 10000 loci, between 8000 and 15000 loci, between 8000 and 20000 loci, between 8000 and 25000 loci, between 8000 and 30000 loci, between 8000 and 35000 loci, between 8000 and 40000 loci, between 8000 and 50000 loci, between 10000 and 15000 loci, between 10000 and 20000 loci, between 10000 and 25000 loci, between 10000 and 30000 loci, between 10000 and 35000 loci, between 10000 and 40000 loci, between 10000 and 50000 loci, between 15000 and 20000 loci, between 15000 and 25000 loci, between 15000 and 30000 loci, between 15000 and 35000 loci, between 15000 and 40000 loci, between 15000 and 50000 loci, between 20000 and 25000 loci, between 20000 and 30000 loci, between 20000 and 35000 loci, between 20000 and 40000 loci, between 20000 and 50000 loci, between 25000 and 30000 loci, between 25000 and 35000 loci, between 25000 and 40000 loci, between 25000 and 50000 loci, between 30000 and 35000 loci, between 30000 and 40000 loci, between 30000 and 50000 loci, between 35000 and 40000 loci, between 35000 and 50000 loci, or between 40000 and 50000 loci.

In some aspects, the one or more gene loci comprise between 10000 and 20000 loci, between 10000 and 40000 loci, between 10000 and 60000 loci, between 10000 and 80000 loci, between 10000 and 100000 loci, between 100000 and 150000 loci, between 10000 and 200000 loci, between 10000 and 250000 loci, between 10000 and 300000 loci, between 10000 and 350000 loci, between 10000 and 400000 loci, between 10000 and 450000 loci, between 10000 and 500000 loci, between 20000 and 40000 loci, between 20000 and 60000 loci, between 20000 and 80000 loci, between 20000 and 100000 loci, between 20000 and 150000 loci, between 20000 and 200000 loci, between 20000 and 250000 loci, between 20000 and 300000 loci, between 20000 and 350000 loci, between 20000 and 400000 loci, between 20000 and 500000 loci, between 40000 and 60000 loci, between 40000 and 80000 loci, between 40000 and 100000 loci, between 40000 and 150000 loci, between 40000 and 200000 loci, between 40000 and 250000 loci, between 40000 and 30000 loci, between 40000 and 350000 loci, between 40000 and 400000 loci, between 40000 and 500000 loci, between 60000 and 80000 loci, between 60000 and 100000 loci, between 60000 and 150000 loci, between 60000 and 200000 loci, between 60000 and 250000 loci, between 60000 and 300000 loci, between 60000 and 350000 loci, between 60000 and 400000 loci, between 60000 and 500000 loci, between 80000 and 100000 loci, between 80000 and 150000 loci, between 80000 and 200000 loci, between 80000 and 250000 loci, between 8000 and 30000 loci, between 8000 and 35000 loci, between 8000 and 40000 loci, between 80000 and 500000 loci, between 100000 and 150000 loci, between 100000 and 200000 loci, between 100000 and 250000 loci, between 100000 and 300000 loci, between 100000 and 350000 loci, between 100000 and 400000 loci, between 100000 and 500000 loci, between 150000 and 200000 loci, between 150000 and 250000 loci, between 150000 and 300000 loci, between 150000 and 350000 loci, between 150000 and 400000 loci, between 150000 and 500000 loci, between 200000 and 250000 loci, between 200000 and 300000 loci, between 200000 and 350000 loci, between 200000 and 400000 loci, between 200000 and 500000 loci, between 250000 and 300000 loci, between 250000 and 350000 loci, between 250000 and 400000 loci, between 250000 and 500000 loci, between 300000 and 350000 loci, between 300000 and 400000 loci, between 300000 and 500000 loci, between 305000 and 400000 loci, between 350000 and 500000 loci, or between 400000 and 500000 loci.

In some instances, the disclosed methods may be used to identify variants in the ABL1, ACVRIB, AKT1, AKT2, AKT3, ALK, ALOX12B, AMER1, APC, AR, ARAF, ARFRP1, ARIDIA, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKNIA, CDKNIB, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNFIA, HRAS, HSD3B1, ID3, IDH1, IDH2, IGFIR, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCDILG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKARIA, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCIL1, WT1, XPO1, XRCC2, ZNF217, or ZNF703 gene locus, or any combination thereof.

In some instances, the disclosed methods may be used to identify variants in the ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, or VEGFB gene locus, or any combination thereof.

### Target Capture Reagents

The methods described herein may comprise contacting a nucleic acid library with a plurality of target capture reagents in order to select and capture a plurality of specific target sequences (e.g., gene sequences or fragments thereof) for analysis. In some instances, a target capture reagent (i.e., a molecule which can bind to and thereby allow capture of a target molecule) is used to select the subject intervals to be analyzed. For example, a target capture reagent can be a bait molecule, e.g., a nucleic acid molecule (e.g., a DNA molecule or RNA molecule) which can hybridize to (i.e., is complementary to) a target molecule, and thereby allows capture of the target nucleic acid. In some instances, the target capture reagent, e.g., a bait molecule (or bait sequence), is a capture oligonucleotide (or capture probe). In some instances, the target nucleic acid is a genomic DNA molecule, an RNA molecule, a cDNA molecule derived from an RNA molecule, a microsatellite DNA sequence, and the like. In some instances, the target capture reagent is suitable for solution-phase hybridization to the target. In some instances, the target capture reagent is suitable for solid-phase hybridization to the target. In some instances, the target capture reagent is suitable for both solution-phase and solid-phase hybridization to the target. The design and construction of target capture reagents is described in more detail in, e.g., International Patent Application Publication No. WO 2020/236941, the entire content of which is incorporated herein by reference.

The methods described herein provide for optimized sequencing of a large number of genomic loci (e.g., genes or gene products (e.g., mRNA), microsatellite loci, etc.) from samples (e.g., cancerous tissue specimens, liquid biopsy samples, and the like) from one or more subjects by the appropriate selection of target capture reagents to select the target nucleic acid molecules to be sequenced. In some instances, a target capture reagent may hybridize to a specific target locus, e.g., a specific target gene locus or fragment thereof. In some instances, a target capture reagent may hybridize to a specific group of target loci, e.g., a specific group of gene loci or fragments thereof. In some instances, a plurality of target capture reagents comprising a mix of target-specific and/or group-specific target capture reagents may be used.

In some instances, the number of target capture reagents (e.g., bait molecules) in the plurality of target capture reagents (e.g., a bait set) contacted with a nucleic acid library to capture a plurality of target sequences for nucleic acid sequencing is greater than 10, greater than 50, greater than 100, greater than 200, greater than 300, greater than 400, greater than 500, greater than 600, greater than 700, greater than 800, greater than 900, greater than 1,000, greater than 1,250, greater than 1,500, greater than 1,750, greater than 2,000, greater than 3,000, greater than 4,000, greater than 5,000, greater than 10,000, greater than 25,000, or greater than 50,000.

In some instances, the overall length of the target capture reagent sequence can be between about 70 nucleotides and 1000 nucleotides. In one instance, the target capture reagent length is between about 100 and 300 nucleotides, 110 and 200 nucleotides, or 120 and 170 nucleotides, in length. In addition to those mentioned above, intermediate oligonucleotide lengths of about 70, 80, 90, 100, 110, 120, 130, 140, 150, 160, 170, 180, 190, 200, 210, 220, 230, 240, 250, 300, 400, 500, 600, 700, 800, and 900 nucleotides in length can be used in the methods described herein. In some embodiments, oligonucleotides of about 70, 80, 90, 100, 110, 120, 130, 140, 150, 160, 170, 180, 190, 200, 210, 220, or 230 bases can be used.

In some instances, each target capture reagent sequence can include: (i) a target-specific capture sequence (e.g., a gene locus or microsatellite locus-specific complementary sequence), (ii) an adapter, primer, barcode, and/or unique molecular identifier sequence, and (iii) universal tails on one or both ends. As used herein, the term “target capture reagent” can refer to the target-specific target capture sequence or to the entire target capture reagent oligonucleotide including the target-specific target capture sequence.

In some instances, the target-specific capture sequences in the target capture reagents are between about 40 nucleotides and 1000 nucleotides in length. In some instances, the target-specific capture sequence is between about 70 nucleotides and 300 nucleotides in length. In some instances, the target-specific sequence is between about 100 nucleotides and 200 nucleotides in length. In yet other instances, the target-specific sequence is between about 120 nucleotides and 170 nucleotides in length, typically 120 nucleotides in length. Intermediate lengths in addition to those mentioned above also can be used in the methods described herein, such as target-specific sequences of about 40, 50, 60, 70, 80, 90, 100, 110, 120, 130, 140, 150, 160, 170, 180, 190, 200, 210, 220, 230, 240, 250, 300, 400, 500, 600, 700, 800, and 900 nucleotides in length, as well as target-specific sequences of lengths between the above-mentioned lengths.

In some instances, the target capture reagent may be designed to select a subject interval containing one or more rearrangements, e.g., an intron containing a genomic rearrangement. In such instances, the target capture reagent is designed such that repetitive sequences are masked to increase the selection efficiency. In those instances where the rearrangement has a known juncture sequence, complementary target capture reagents can be designed to recognize the juncture sequence to increase the selection efficiency.

In some instances, the disclosed methods may comprise the use of target capture reagents designed to capture two or more different target categories, each category having a different target capture reagent design strategy. In some instances, the hybridization-based capture methods and target capture reagent compositions disclosed herein may provide for the capture and homogeneous coverage of a set of target sequences, while minimizing coverage of genomic sequences outside of the targeted set of sequences. In some instances, the target sequences may include the entire exome of genomic DNA or a selected subset thereof. In some instances, the target sequences may include, e.g., a large chromosomal region (e.g., a whole chromosome arm). The methods and compositions disclosed herein provide different target capture reagents for achieving different sequencing depths and patterns of coverage for complex sets of target nucleic acid sequences.

Typically, DNA molecules are used as target capture reagent sequences, although RNA molecules can also be used. In some instances, a DNA molecule target capture reagent can be single stranded DNA (ssDNA) or double-stranded DNA (dsDNA). In some instances, an RNA-DNA duplex is more stable than a DNA-DNA duplex and therefore provides for potentially better capture of nucleic acids.

In some instances, the disclosed methods comprise providing a selected set of nucleic acid molecules (e.g., a library catch) captured from one or more nucleic acid libraries. For example, the method may comprise: providing one or a plurality of nucleic acid libraries, each comprising a plurality of nucleic acid molecules (e.g., a plurality of target nucleic acid molecules and/or reference nucleic acid molecules) extracted from one or more samples from one or more subjects; contacting the one or a plurality of libraries (e.g., in a solution-based hybridization reaction) with one, two, three, four, five, or more than five pluralities of target capture reagents (e.g., oligonucleotide target capture reagents) to form a hybridization mixture comprising a plurality of target capture reagent/nucleic acid molecule hybrids; separating the plurality of target capture reagent/nucleic acid molecule hybrids from said hybridization mixture, e.g., by contacting said hybridization mixture with a binding entity that allows for separation of said plurality of target capture reagent/nucleic acid molecule hybrids from the hybridization mixture, thereby providing a library catch (e.g., a selected or enriched subgroup of nucleic acid molecules from the one or a plurality of libraries).

In some instances, the disclosed methods may further comprise amplifying the library catch (e.g., by performing PCR). In other instances, the library catch is not amplified.

In some instances, the target capture reagents can be part of a kit which can optionally comprise instructions, standards, buffers or enzymes or other reagents.

### Hybridization Conditions

As noted above, the methods disclosed herein may include the step of contacting the library (e.g., the nucleic acid library) with a plurality of target capture reagents to provide a selected library target nucleic acid sequences (i.e., the library catch). The contacting step can be effected in, e.g., solution-based hybridization. In some instances, the method includes repeating the hybridization step for one or more additional rounds of solution-based hybridization. In some instances, the method further includes subjecting the library catch to one or more additional rounds of solution-based hybridization with the same or a different collection of target capture reagents.

In some instances, the contacting step is effected using a solid support, e.g., an array. Suitable solid supports for hybridization are described in, e.g., Albert, T. J. et al. (2007) Nat. Methods 4(11):903-5; Hodges, E. et al. (2007) Nat. Genet. 39(12):1522-7; and Okou, D. T. et al. (2007) Nat. Methods 4(11):907-9, the contents of which are incorporated herein by reference in their entireties.

Hybridization methods that can be adapted for use in the methods herein are described in the art, e.g., as described in International Patent Application Publication No. WO 2012/092426. Methods for hybridizing target capture reagents to a plurality of target nucleic acids are described in more detail in, e.g., International Patent Application Publication No. WO 2020/236941, the entire content of which is incorporated herein by reference.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subject interval in the sample and thereby determine, e.g., gene allele sequences at a plurality of gene loci. “Next-generation sequencing” (or “NGS”) as used herein may also be referred to as “massively parallel sequencing” (or “MPS”), and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche/454 Genome Sequencer (GS) FLX System, Illumina/Solexa Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLID) system, Polonator's G.007 system, Helicos BioSciences' HeliScope Gene Sequencing system, or Pacific Biosciences' PacBio® RS platform. In some instances, sequencing may comprise Illumina MiSeq™ sequencing. In some instances, sequencing may comprise Illumina HiSeq® sequencing. In some instances, sequencing may comprise Illumina NovaSeq® sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Alignment

Alignment is the process of matching a read with a location, e.g., a genomic location or locus. In some instances, NGS reads may be aligned to a known reference sequence (e.g., a wild-type sequence). In some instances, NGS reads may be assembled de novo. Methods of sequence alignment for NGS reads are described in, e.g., Trapnell, C. and Salzberg, S. L. Nature Biotech., 2009, 27:455-457. Examples of de novo sequence assemblies are described in, e.g., Warren R., et al., Bioinformatics, 2007, 23:500-501; Butler, J. et al., Genome Res., 2008, 18:810-820; and Zerbino, D. R. and Birney, E., Genome Res., 2008, 18:821-829. Optimization of sequence alignment is described in the art, e.g., as set out in International Patent Application Publication No. WO 2012/092426. Additional description of sequence alignment methods is provided in, e.g., International Patent Application Publication No. WO 2020/236941, the entire content of which is incorporated herein by reference.

Misalignment (e.g., the placement of base-pairs from a short read at incorrect locations in the genome), e.g., misalignment of reads due to sequence context (e.g., the presence of repetitive sequence) around an actual cancer mutation can lead to reduction in sensitivity of mutation detection, can lead to a reduction in sensitivity of mutation detection, as reads for the alternate allele may be shifted off the histogram peak of alternate allele reads. Other examples of sequence context that may cause misalignment include short-tandem repeats, interspersed repeats, low complexity regions, insertions-deletions (indels), and paralogs. If the problematic sequence context occurs where no actual mutation is present, misalignment may introduce artifactual reads of “mutated” alleles by placing reads of actual reference genome base sequences at the wrong location. Because mutation-calling algorithms for multigene analysis should be sensitive to even low-abundance mutations, sequence misalignments may increase false positive discovery rates and/or reduce specificity.

In some instances, the methods and systems disclosed herein may integrate the use of multiple, individually-tuned, alignment methods or algorithms to optimize base-calling performance in sequencing methods, particularly in methods that rely on massively parallel sequencing (MPS) of a large number of diverse genetic events at a large number of diverse genomic loci. In some instances, the disclosed methods and systems may comprise the use of one or more global alignment algorithms. In some instances, the disclosed methods and systems may comprise the use of one or more local alignment algorithms. Examples of alignment algorithms that may be used include, but are not limited to, the Burrows-Wheeler Alignment (BWA) software bundle (see, e.g., Li, et al. (2009), “Fast and Accurate Short Read Alignment with Burrows-Wheeler Transform”, Bioinformatics 25:1754-60; Li, et al. (2010), Fast and Accurate Long-Read Alignment with Burrows-Wheeler Transform”, Bioinformatics epub. PMID: 20080505), the Smith-Waterman algorithm (see, e.g., Smith, et al. (1981), “Identification of Common Molecular Subsequences”, J. Molecular Biology 147(1):195-197), the Striped Smith-Waterman algorithm (see, e.g., Farrar (2007), “Striped Smith-Waterman Speeds Database Searches Six Times Over Other SIMD Implementations”, Bioinformatics 23(2):156-161), the Needleman-Wunsch algorithm (Needleman, et al. (1970) “A General Method Applicable to the Search for Similarities in the Amino Acid Sequence of Two Proteins”, J. Molecular Biology 48(3):443-53), or any combination thereof.

In some instances, the methods and systems disclosed herein may also comprise the use of a sequence assembly algorithm, e.g., the Arachne sequence assembly algorithm (see, e.g., Batzoglou, et al. (2002), “ARACHNE: A Whole-Genome Shotgun Assembler”, Genome Res. 12:177-189).

In some instances, the alignment method used to analyze sequence reads is not individually customized or tuned for detection of different variants (e.g., point mutations, insertions, deletions, and the like) at different genomic loci. In some instances, different alignment methods are used to analyze reads that are individually customized or tuned for detection of at least a subset of the different variants detected at different genomic loci. In some instances, different alignment methods are used to analyze reads that are individually customized or tuned to detect each different variant at different genomic loci. In some instances, tuning can be a function of one or more of: (i) the genetic locus (e.g., gene loci, microsatellite locus, or other subject interval) being sequenced, (ii) the tumor type associated with the sample, (iii) the variant being sequenced, or (iv) a characteristic of the sample or the subject. The selection or use of alignment conditions that are individually tuned to a number of specific subject intervals to be sequenced allows optimization of speed, sensitivity, and specificity. The method is particularly effective when the alignment of reads for a relatively large number of diverse subject intervals are optimized.

In some instances, the method includes the use of an alignment method optimized for rearrangements in combination with other alignment methods optimized for subject intervals not associated with rearrangements.

In some instances, the methods disclosed herein further comprise selecting or using an alignment method for analyzing, e.g., aligning, a sequence read, wherein said alignment method is a function of, is selected responsive to, or is optimized for, one or more of: (i) tumor type, e.g., the tumor type in the sample; (ii) the location (e.g., a gene locus) of the subject interval being sequenced; (iii) the type of variant (e.g., a point mutation, insertion, deletion, substitution, copy number variation (CNV), rearrangement, or fusion) in the subject interval being sequenced; (iv) the site (e.g., nucleotide position) being analyzed; (v) the type of sample (e.g., a sample described herein); and/or (vi) adjacent sequence(s) in or near the subject interval being evaluated (e.g., according to the expected propensity thereof for misalignment of the subject interval due to, e.g., the presence of repeated sequences in or near the subject interval).

In some instances, the methods disclosed herein allow for the rapid and efficient alignment of troublesome reads, e.g., a read having a rearrangement. Thus, in some instances where a read for a subject interval comprises a nucleotide position with a rearrangement, e.g., a translocation, the method can comprise using an alignment method that is appropriately tuned and that includes: (i) selecting a rearrangement reference sequence for alignment with a read, wherein said rearrangement reference sequence aligns with a rearrangement (in some instances, the reference sequence is not identical to the genomic rearrangement); and (ii) comparing, e.g., aligning, a read with said rearrangement reference sequence.

In some instances, alternative methods may be used to align troublesome reads. These methods are particularly effective when the alignment of reads for a relatively large number of diverse subject intervals is optimized. By way of example, a method of analyzing a sample can comprise: (i) performing a comparison (e.g., an alignment comparison) of a read using a first set of parameters (e.g., using a first mapping algorithm, or by comparison with a first reference sequence), and determining if said read meets a first alignment criterion (e.g., the read can be aligned with said first reference sequence, e.g., with less than a specific number of mismatches); (ii) if said read fails to meet the first alignment criterion, performing a second alignment comparison using a second set of parameters, (e.g., using a second mapping algorithm, or by comparison with a second reference sequence); and (iii) optionally, determining if said read meets said second criterion (e.g., the read can be aligned with said second reference sequence, e.g., with less than a specific number of mismatches), wherein said second set of parameters comprises use of, e.g., said second reference sequence, which, compared with said first set of parameters, is more likely to result in an alignment with a read for a variant (e.g., a rearrangement, insertion, deletion, or translocation).

In some instances, the alignment of sequence reads in the disclosed methods may be combined with a mutation calling method as described elsewhere herein. As discussed herein, reduced sensitivity for detecting actual mutations may be addressed by evaluating the quality of alignments (manually or in an automated fashion) around expected mutation sites in the genes or genomic loci (e.g., gene loci) being analyzed. In some instances, the sites to be evaluated can be obtained from databases of the human genome (e.g., the HG19 human reference genome) or cancer mutations (e.g., COSMIC). Regions that are identified as problematic can be remedied with the use of an algorithm selected to give better performance in the relevant sequence context, e.g., by alignment optimization (or re-alignment) using slower, but more accurate alignment algorithms such as Smith-Waterman alignment. In cases where general alignment algorithms cannot remedy the problem, customized alignment approaches may be created by, e.g., adjustment of maximum difference mismatch penalty parameters for genes with a high likelihood of containing substitutions; adjusting specific mismatch penalty parameters based on specific mutation types that are common in certain tumor types (e.g. C→T in melanoma); or adjusting specific mismatch penalty parameters based on specific mutation types that are common in certain sample types (e.g. substitutions that are common in FFPE).

Reduced specificity (increased false positive rate) in the evaluated subject intervals due to misalignment can be assessed by manual or automated examination of all mutation calls in the sequencing data. Those regions found to be prone to spurious mutation calls due to misalignment can be subjected to alignment remedies as discussed above. In cases where no algorithmic remedy is found possible, “mutations” from the problem regions can be classified or screened out from the panel of targeted loci.

### Alignment of Methyl-Seq Sequence Reads

In some instances, the methods may include the use of an alignment method optimized for aligning sequence reads for DNA that has been converted using, e.g., a bisulfite reaction, to convert unmethylated cytosine residues to uracil (which is interpreted as a thymine in sequencing results). In some instances, sequence reads may be aligned to two genomes in silico, e.g., converted and unconverted versions of the reference genome, using such alignment tools. Methylation occurs primarily at CpG sites, but may also occur less frequently at non-CpG sites (e.g., CHG or CHH sites).

In some instances, the sequence read data may be obtained using a nucleic acid sequencing method comprising the use of a bisulfite- or enzymatic-conversion reaction (e.g., during library preparation) to convert non-methylated cytosine to uracil (see, e.g., Li, et al. (2011), “DNA Methylation Detection: Bisulfite Genomic Sequencing Analysis”, Methods Mol. Biol. 791:11-21).

In some instances, the sequence read data may be obtained using a nucleic acid sequencing method comprising the use of alternative chemical and/or enzymatic reactions (e.g., during library preparation) to convert non-methylated cytosine to uracil (or to convert methylated cytosine to dihydrouracil). For example, enzymatic deamination of non-methylated cytosine using APOBEC to form uracil can be performed using, e.g., the Enzymatic Methyl-seq Kit from New England BioLabs (Ipswich, MA) which uses prior treatment with ten-eleven translocation methylcytosine dioxygenase 2 (TET2) to oxidize 5-mC and 5-hmC, thereby providing greater protection of the methylated cytosine from deamination by APOBEC). Liu, et al. (2019) recently described a bisulfite-free and base-level-resolution sequencing-based method, TET-Assisted Pyridine borane Sequencing (TAPS), for detection of 5mC and 5hmC. The method combines ten-eleven translocation methylcytosine dioxygenase (TET)-mediated oxidation of 5mC and 5hmC to 5-carboxylcytosine (5caC) with pyridine borane reduction of 5caC to dihydrouracil (DHU). Subsequent PCR amplification converts DHU to thymine, thereby enabling conversion of methylated cytosines to thymine (Liu, et al. (2019), “Bisulfite-Free Direct Detection of 5-Methylcytosine and 5-Hydroxymethylcytosine at Base Resolution”, Nature Biotechnology, vol. 37, pp. 424-429).

In some instances, the sequence read data may be obtained using a nucleic acid sequencing method comprising the use of Methylated DNA Immunoprecipitation (MeDIP).

Examples of alignment tools optimized for aligning sequence reads for converted DNA include, but are not limited to, NovoAlign (Novocraft Technologies, Selangor, Malaysia), and the Bismark tool (Krueger, et al. (2011), “Bismark: A Flexible Aligner and Methylation Caller for Bisulfite-Seq Applications”, Bioinformatics 27 (11): 1571-1572).

### Mutation Calling

Base calling refers to the raw output of a sequencing device, e.g., the determined sequence of nucleotides in an oligonucleotide molecule. Mutation calling refers to the process of selecting a nucleotide value, e.g., A, G, T, or C, for a given nucleotide position being sequenced. Typically, the sequence reads (or base calling) for a position will provide more than one value, e.g., some reads will indicate a T and some will indicate a G. Mutation calling is the process of assigning a correct nucleotide value, e.g., one of those values, to the sequence. Although it is referred to as “mutation” calling, it can be applied to assign a nucleotide value to any nucleotide position, e.g., positions corresponding to mutant alleles, wild-type alleles, alleles that have not been characterized as either mutant or wild-type, or to positions not characterized by variability.

In some instances, the disclosed methods may comprise the use of customized or tuned mutation calling algorithms or parameters thereof to optimize performance when applied to sequencing data, particularly in methods that rely on massively parallel sequencing (MPS) of a large number of diverse genetic events at a large number of diverse genomic loci (e.g., gene loci, microsatellite regions, etc.) in samples, e.g., samples from a subject having cancer. Optimization of mutation calling is described in the art, e.g., as set out in International Patent Application Publication No. WO 2012/092426.

Methods for mutation calling can include one or more of the following: making independent calls based on the information at each position in the reference sequence (e.g., examining the sequence reads; examining the base calls and quality scores; calculating the probability of observed bases and quality scores given a potential genotype; and assigning genotypes (e.g., using Bayes' rule)); removing false positives (e.g., using depth thresholds to reject SNPs with read depth much lower or higher than expected; local realignment to remove false positives due to small indels); and performing linkage disequilibrium (LD)/imputation-based analysis to refine the calls.

Equations used to calculate the genotype likelihood associated with a specific genotype and position are described in, e.g., Li, H. and Durbin, R. Bioinformatics, 2010; 26(5):589-95. The prior expectation for a particular mutation in a certain cancer type can be used when evaluating samples from that cancer type. Such likelihood can be derived from public databases of cancer mutations, e.g., Catalogue of Somatic Mutation in Cancer (COSMIC), HGMD (Human Gene Mutation Database), The SNP Consortium, Breast Cancer Mutation Data Base (BIC), and Breast Cancer Gene Database (BCGD).

Examples of LD/imputation based analysis are described in, e.g., Browning, B. L. and Yu, Z. Am. J. Hum. Genet. 2009, 85(6):847-61. Examples of low-coverage SNP calling methods are described in, e.g., Li, Y., et al., Annu. Rev. Genomics Hum. Genet. 2009, 10:387-406.

After alignment, detection of substitutions can be performed using a mutation calling method (e.g., a Bayesian mutation calling method) which is applied to each base in each of the subject intervals, e.g., exons of a gene or other locus to be evaluated, where presence of alternate alleles is observed. This method will compare the probability of observing the read data in the presence of a mutation with the probability of observing the read data in the presence of base-calling error alone. Mutations can be called if this comparison is sufficiently strongly supportive of the presence of a mutation.

An advantage of a Bayesian mutation detection approach is that the comparison of the probability of the presence of a mutation with the probability of base-calling error alone can be weighted by a prior expectation of the presence of a mutation at the site. If some reads of an alternate allele are observed at a frequently mutated site for the given cancer type, then presence of a mutation may be confidently called even if the amount of evidence of mutation does not meet the usual thresholds. This flexibility can then be used to increase detection sensitivity for even rarer mutations/lower purity samples, or to make the test more robust to decreases in read coverage. The likelihood of a random base-pair in the genome being mutated in cancer is ~1e−6. The likelihood of specific mutations occurring at many sites in, for example, a typical multigenic cancer genome panel can be orders of magnitude higher. These likelihoods can be derived from public databases of cancer mutations (e.g., COSMIC).

Indel calling is a process of finding bases in the sequencing data that differ from the reference sequence by insertion or deletion, typically including an associated confidence score or statistical evidence metric. Methods of indel calling can include the steps of identifying candidate indels, calculating genotype likelihood through local re-alignment, and performing LD-based genotype inference and calling. Typically, a Bayesian approach is used to obtain potential indel candidates, and then these candidates are tested together with the reference sequence in a Bayesian framework.

Algorithms to generate candidate indels are described in, e.g., McKenna, A., et al., Genome Res. 2010; 20(9):1297-303; Ye, K., et al., Bioinformatics, 2009; 25(21):2865-71; Lunter, G., and Goodson, M., Genome Res. 2011; 21(6):936-9; and Li, H., et al. (2009), Bioinformatics 25(16):2078-9.

Methods for generating indel calls and individual-level genotype likelihoods include, e.g., the Dindel algorithm (Albers, C. A., et al., Genome Res. 2011; 21(6):961-73). For example, the Bayesian EM algorithm can be used to analyze the reads, make initial indel calls, and generate genotype likelihoods for each candidate indel, followed by imputation of genotypes using, e.g., QCALL (Le S. Q. and Durbin R. Genome Res. 2011; 21(6):952-60). Parameters, such as prior expectations of observing the indel can be adjusted (e.g., increased or decreased), based on the size or location of the indels.

Methods have been developed that address limited deviations from allele frequencies of 50% or 100% for the analysis of cancer DNA. (see, e.g., SNVMix-Bioinformatics. 2010 Mar. 15; 26(6): 730-736.) Methods disclosed herein, however, allow consideration of the possibility of the presence of a mutant allele at frequencies (or allele fractions) ranging from 1% to 100% (i.e., allele fractions ranging from 0.01 to 1.0), and especially at levels lower than 50%. This approach is particularly important for the detection of mutations in, for example, low-purity FFPE samples of natural (multi-clonal) tumor DNA.

In some instances, the mutation calling method used to analyze sequence reads is not individually customized or fine-tuned for detection of different mutations at different genomic loci. In some instances, different mutation calling methods are used that are individually customized or fine-tuned for at least a subset of the different mutations detected at different genomic loci. In some instances, different mutation calling methods are used that are individually customized or fine-tuned for each different mutant detected at each different genomic loci. The customization or tuning can be based on one or more of the factors described herein, e.g., the type of cancer in a sample, the gene or locus in which the subject interval to be sequenced is located, or the variant to be sequenced. This selection or use of mutation calling methods individually customized or fine-tuned for a number of subject intervals to be sequenced allows for optimization of speed, sensitivity and specificity of mutation calling.

In some instances, a nucleotide value is assigned for a nucleotide position in each of X unique subject intervals using a unique mutation calling method, and X is at least 2, at least 3, at least 4, at least 5, at least 10, at least 15, at least 20, at least 30, at least 40, at least 50, at least 60, at least 70, at least 80, at least 90, at least 100, at least 200, at least 300, at least 400, at least 500, at least 1000, at least 1500, at least 2000, at least 2500, at least 3000, at least 3500, at least 4000, at least 4500, at least 5000, or greater. The calling methods can differ, and thereby be unique, e.g., by relying on different Bayesian prior values.

In some instances, assigning said nucleotide value is a function of a value which is or represents the prior (e.g., literature) expectation of observing a read showing a variant, e.g., a mutation, at said nucleotide position in a tumor of type.

In some instances, the method comprises assigning a nucleotide value (e.g., calling a mutation) for at least 10, 20, 40, 50, 60, 70, 80, 90, 100, 200, 300, 400, 500, 600, 700, 800, 900, or 1,000 nucleotide positions, wherein each assignment is a function of a unique value (as opposed to the value for the other assignments) which is or represents the prior (e.g., literature) expectation of observing a read showing a variant, e.g., a mutation, at said nucleotide position in a tumor of type.

In some instances, assigning said nucleotide value is a function of a set of values which represent the probabilities of observing a read showing said variant at said nucleotide position if the variant is present in the sample at a specified frequency (e.g., 1%, 5%, 10%, etc.) and/or if the variant is absent (e.g., observed in the reads due to base-calling error alone).

In some instances, the mutation calling methods described herein can include the following: (a) acquiring, for a nucleotide position in each of said X subject intervals: (i) a first value which is or represents the prior (e.g., literature) expectation of observing a read showing a variant, e.g., a mutation, at said nucleotide position in a tumor of type X; and (ii) a second set of values which represent the probabilities of observing a read showing said variant at said nucleotide position if the variant is present in the sample at a frequency (e.g., 1%, 5%, 10%, etc.) and/or if the variant is absent (e.g., observed in the reads due to base-calling error alone); and (b) responsive to said values, assigning a nucleotide value (e.g., calling a mutation) from said reads for each of said nucleotide positions by weighing, e.g., by a Bayesian method described herein, the comparison among the values in the second set using the first value (e.g., computing the posterior probability of the presence of a mutation), thereby analyzing said sample.

Additional description of exemplary nucleic acid sequencing methods, mutation calling methods, and methods for analysis of genetic variants is provided in, e.g., U.S. Pat. Nos. 9,340,830, 9,792,403, 11,136,619, 11,118,213, and International Patent Application Publication No. WO 2020/236941, the entire contents of each of which is incorporated herein by reference.

### Methylation Status Calling

In some instances, the methods described herein may comprise the use of a methylation status calling method, e.g., to call the methylation status of the CpG sites based on the sequence reads and fragments (complementary pairs of forward and reverse sequence reads) derived from DNA that has been subjected to a chemical or enzymatic conversion reaction, e.g., to convert unmethylated cytosine residues to uracil (which is interpreted as a thymine in sequencing results). Examples of such methylation status calling tools include, but are not limited to, the Bismark tool (Krueger, et al. (2011), “Bismark: A Flexible Aligner and Methylation Caller for Bisulfite-Seq Applications”, Bioinformatics 27(11):1571-1572), TARGOMICS (Garinet, et al. (2017), “Calling Chromosome Alterations, DNA Methylation Statuses, and Mutations in Tumors by Simple Targeted Next-Generation Sequencing—A Solution for Transferring Integrated Pangenomic Studies into Routine Practice?”, J. Molecular Diagnostics 19(5):776-787), Bicycle (Grana, et al. (2018) “Bicycle: A Bioinformatics Pipeline to Analyze Bisulfite Sequencing Data”, Bioinformatics 34(8):1414-5), SMAP (Gao, et al. (2015), “SMAP: A Streamlined Methylation Analysis Pipeline for Bisulfite Sequencing”, Gigascience 4:29), and MeDUSA (Wilson, et al. (2016), “Computational Analysis and Integration of MeDIP-Seq Methylome Data”, in: Kulski J K, editor, Next Generation Sequencing: Advances, Applications and Challenges. Rijeka: InTech, p. 153-69). See also, Rauluseviciute, et al. (2019), “DNA Methylation Data by Sequencing: Experimental Approaches and Recommendations for Tools and Pipelines for Data Analysis”, Clinical Epigenetics 11:193.

### Microbiome Profiling

In some instances, the methods described herein comprise microbiome profiling. Microbiome profiling data may be used as training data and/or test data as described herein. Sequencing methods described herein may be adapted using methods known in the art for microbiome profiling. Sequencing methods for microbiome profiling may include 16s ribosomal RNA sequencing, metagenomic sequencing, or metatranscriptomic sequencing. 16s ribosomal sequencing comprises sequencing the 16s ribosomal gene in DNA extracted from a sample and using the data to identify and classify microbacterium species in the sample. Metagenomic sequencing comprises sequencing all genetic material in a sample and using the data to identify and classify microbacterium species. Metatranscriptomic sequencing comprises sequencing RNA transcripts from a sample to study active gene expression by microbacterium species in the sample. In some instances, metabolomic techniques can be used to analyze metabolites produced by microbacterium species in a sample.

### Systems

Also disclosed herein are systems designed to implement any of the disclosed methods for generating a feature group for distinguishing a first disease characterization class from a second disease characterization class, classifying a first disease characterization class from a second disease characterization in a sample from a subject, and for designing nucleic acid baits. The systems may comprise, e.g., one or more processors, and a memory unit communicatively coupled to the one or more processors and configured to store instructions that, when executed by the one or more processors, cause the system to: receive, at one or more processors, training data comprising values for features for a plurality of training samples comprising a disease characterization subclass label, wherein the disease characterization subclass label represents one of a plurality of disease characterization subclasses to which the training sample belongs to and wherein the plurality of training samples represents a plurality of disease characterization subclass labels; select, by the one or more processors, pairwise subclass representative features that distinguish the disease characterization subclasses from other disease characterization subclasses in the plurality of disease characterization subclasses; select, by one or more processors, a first subset of the plurality of the disease characterization subclass labels as belonging to the first disease characterization class and a second subset of the plurality of the disease characterization subclass labels as belonging to the second disease characterization class; select, by one or more processors, a subset of pairwise subclass representative features from the pairwise subclass representative features, wherein the subset of pairwise subclass representative features distinguish disease characterization subclasses from within a common disease characterization class; generate, by the one or more processors, a feature group by removing the subset of pairwise subclass representative features from the pairwise subclass representative features.

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for generating a feature group for distinguishing a first disease class from a second disease class using training data collected from any of a variety of samples as described herein (e.g., a tissue sample, biopsy sample, hematological sample, or liquid biopsy sample derived from the subject). In some instances, the discloses systmtes may be used for classifying a sample is any of a variety of samples as described herein (e.g., a tissue sample, biopsy sample, hematological sample, or liquid biopsy sample derived from the subject).

In some instances, the plurality of gene loci for which sequencing data is processed to determine the features groups and/or classify as sample as a first disease characterization class or a second disease characterization class may comprise at least 1, 2, 3, 4, 5, 6, 7, 8, 9, 10, 20, 30, 40, 50, 60, 70, 80, 0, 100, 200, 300, 400, 500, 600, 700, 800, 900, 1000, or more than 1000 gene loci (or any number of gene loci within the range of 1 to more than 1000 gene loci).

In some instance, the nucleic acid sequence data is acquired using a next generation sequencing technique (also referred to as a massively parallel sequencing technique) having a read-length of less than 400 bases, less than 300 bases, less than 200 bases, less than 150 bases, less than 100 bases, less than 90 bases, less than 80 bases, less than 70 bases, less than 60 bases, less than 50 bases, less than 40 bases, or less than 30 bases.

In some instances, the determination of a first disease characterization class or second disease characterization class is used to select, initiate, adjust, or terminate a treatment for cancer in the subject (e.g., a patient) from which the sample was derived, as described elsewhere herein.

In some instances, the disclosed systems may further comprise sample processing and library preparation workstations, microplate-handling robotics, fluid dispensing systems, temperature control modules, environmental control chambers, additional data storage modules, data communication modules (e.g., Bluetooth®, WiFi, intranet, or internet communication hardware and associated software), display modules, one or more local and/or cloud-based software packages (e.g., instrument/system control software packages, sequencing data analysis software packages), etc., or any combination thereof. In some instances, the systems may comprise, or be part of, a computer system or computer network as described elsewhere herein.

### Computer Systems and Networks

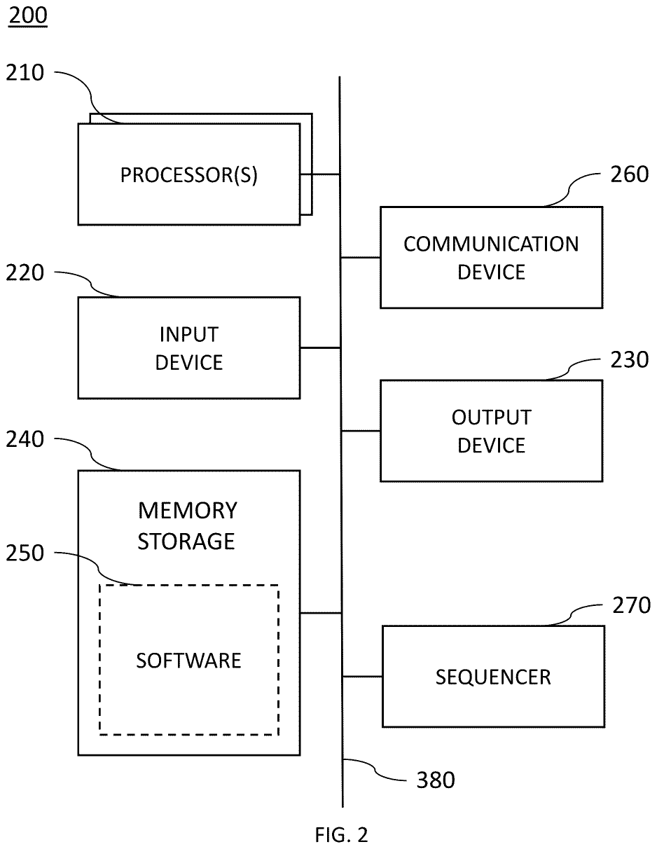

FIG. 2 illustrates an example of a computing device or system in accordance with one embodiment. Device 200 can be a host computer connected to a network. Device 200 can be a client computer or a server. As shown in FIG. 2, device 200 can be any suitable type of microprocessor-based device, such as a personal computer, workstation, server or handheld computing device (portable electronic device) such as a phone or tablet. The device can include, for example, one or more processor(s) 210, input devices 220, output devices 230, memory or storage devices 240, communication devices 260, and nucleic acid sequencers 270. Software 250 residing in memory or storage device 240 may comprise, e.g., an operating system as well as software for executing the methods described herein. Input device 220 and output device 230 can generally correspond to those described herein, and can either be connectable or integrated with the computer.

Input device 220 can be any suitable device that provides input, such as a touch screen, keyboard or keypad, mouse, or voice-recognition device. Output device 230 can be any suitable device that provides output, such as a touch screen, haptics device, or speaker.

Storage 240 can be any suitable device that provides storage (e.g., an electrical, magnetic or optical memory including a RAM (volatile and non-volatile), cache, hard drive, or removable storage disk). Communication device 260 can include any suitable device capable of transmitting and receiving signals over a network, such as a network interface chip or device. The components of the computer can be connected in any suitable manner, such as via a wired media (e.g., a physical system bus 280, Ethernet connection, or any other wire transfer technology) or wirelessly (e.g., Bluetooth®, Wi-Fi®, or any other wireless technology).

Software module 250, which can be stored as executable instructions in storage 240 and executed by processor(s) 210, can include, for example, an operating system and/or the processes that embody the functionality of the methods of the present disclosure (e.g., as embodied in the devices as described herein).

Software module 250 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 940, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 250 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

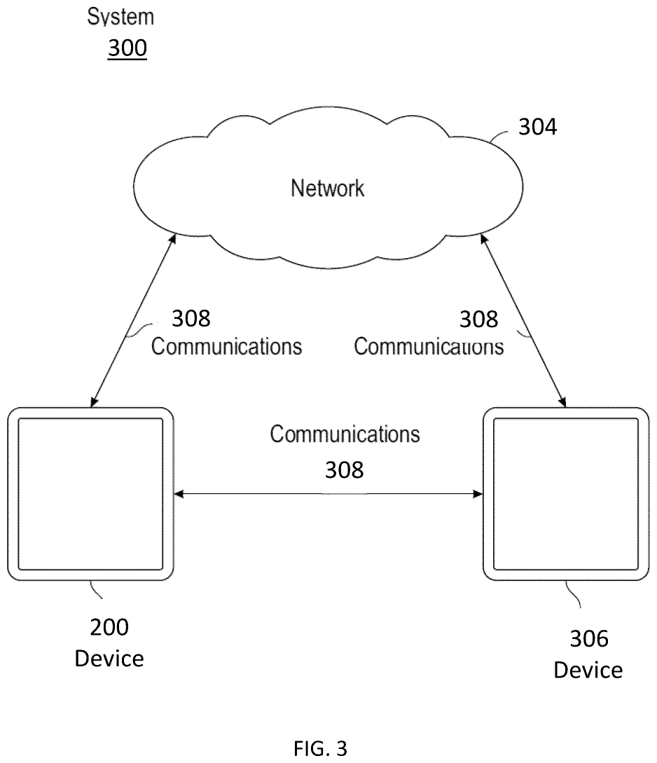

Device 200 may be connected to a network (e.g., network 304, as shown in FIG. 3 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 200 can be implemented using any operating system, e.g., an operating system suitable for operating on the network. Software module 250 can be written in any suitable programming language, such as C, C++, Java, R or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 210.

Device 200 can further include a sequencer 270, which can be any suitable nucleic acid sequencing instrument.

FIG. 3 illustrates an example of a computing system in accordance with one embodiment. In system 300, device 200 (e.g., as described above and illustrated in FIG. 2) is connected to network 304, which is also connected to device 306. In some embodiments, device 306 is a sequencer. Exemplary sequencers can include, without limitation, Roche/454's Genome Sequencer (GS) FLX System, Illumina/Solexa's Genome Analyzer (GA), Illumina's HiSeq® 2500, HiSeq® 3000, HiSeq® 4000 and NovaSeq® 6000 Sequencing Systems, Life/APG's Support Oligonucleotide Ligation Detection (SOLiD) system, Polonator's G.007 system, Helicos BioSciences' HeliScope Gene Sequencing system, Pacific Biosciences' PacBio® RS system, Ultima Genomics UG 100™ platform, or the Illumina NovaSeq X series platform.

Devices 200 and 306 may communicate, e.g., using suitable communication interfaces via network 304, such as a Local Area Network (LAN), Virtual Private Network (VPN), or the Internet. In some embodiments, network 304 can be, for example, the Internet, an intranet, a virtual private network, a cloud network, a wired network, or a wireless network. Devices 300 and 306 may communicate, in part or in whole, via wireless or hardwired communications, such as Ethernet, IEEE 802.11b wireless, or the like. Additionally, devices 200 and 306 may communicate, e.g., using suitable communication interfaces, via a second network, such as a mobile/cellular network. Communication between devices 200 and 306 may further include or communicate with various servers such as a mail server, mobile server, media server, telephone server, and the like. In some embodiments, Devices 200 and 306 can communicate directly (instead of, or in addition to, communicating via network 304), e.g., via wireless or hardwired communications, such as Ethernet, IEEE 802.11b wireless, or the like. In some embodiments, devices 200 and 306 communicate via communications 308, which can be a direct connection or can occur via a network (e.g., network 304).

One or all of devices 200 and 306 generally include logic (e.g., http web server logic) or are programmed to format data, accessed from local or remote databases or other sources of data and content, for providing and/or receiving information via network 304 according to various examples described herein.

### EXEMPLARY IMPLEMENTATIONS

Exemplary implementations of the methods and systems described herein include:

Embodiment 1: The method of generating a feature group for distinguishing a first disease characterization class from a second disease characterization class, comprising: receiving, at one or more processors, training data comprising values for features for a plurality of training samples comprising a disease characterization subclass label, wherein the disease characterization subclass label represents one of a plurality of disease characterization subclasses to which the training sample belongs, and wherein the plurality of training samples represents a plurality of disease characterization subclass labels; selecting, by the one or more processors, pairwise subclass representative features that distinguish the disease characterization subclasses from other disease characterization subclasses in the plurality of disease characterization subclasses; selecting, by one or more processors, a first subset of the plurality of the disease characterization subclass labels as belonging to the first disease characterization class and a second subset of the plurality of the disease characterization subclass labels as belonging to the second disease characterization class; selecting, by one or more processors, a subset of pairwise subclass representative features from the pairwise subclass representative features, wherein the subset of pairwise subclass representative features that distinguish disease characterization subclasses from within a common disease characterization class; generating, by the one or more processors, a feature group by removing the subset of pairwise subclass representative features from the pairwise subclass representative features.

Embodiment 2: The method of embodiment 1, wherein the features are associated with a plurality of subject intervals, a plurality of genes, a plurality of epigenetic states and/or a plurality of genetic alterations.

Embodiment 3: The method of embodiment 1 or 2, wherein the values comprise mRNA expression values, protein expression values, methylation levels, and/or a genetic alteration status.

Embodiment 4: The method of embodiment 1 or 2, wherein the features are associated with a plurality of microbacterium species.

Embodiment 5: The method of embodiment 4, wherein the values comprise an abundance of a microbacterium species in the plurality of microbacterium species.

Embodiment 6: The method of any of embodiments 1-5, wherein the disease characterizing subclasses relate to a pathological status, tissue of origin, or stage of a disease.

Embodiment 7: The method of any of embodiments 1-6, wherein the first disease characterization class and/or second characterization class comprises a cancer characterization.

Embodiment 8: The method of any of embodiments 1-7, wherein the first disease characterization class is cancer, and the second disease characterization class is non-cancer.

Embodiment 9: The method of embodiment 8, wherein the first subset of disease characterization subclasses comprises, colorectal cancer, advanced adenoma, lung cancer, lung adenocarcinoma, lung squamous-cell carcinoma, esophageal cancer, stomach cancer, liver cancer, bile duct cancer, pancreatic cancer, bladder cancer, and ovarian cancer, and the second subset of disease characterization subclasses comprises non-advanced adenoma and non-cancer.

Embodiment 10: The method of any of embodiments 1-9, wherein the first disease characterization class is colorectal cancer, and the second disease characterization class is lung cancer.

Embodiment 11: The method of embodiment 10, wherein the first subset of disease characterization subclasses comprises, colorectal cancer stage I, colorectal cancer stage II, colorectal cancer stage III, colorectal cancer stage IV, and the second subset of disease characterization subclasses comprises lung cancer stage I, lung cancer stage II, lung cancer stage III, lung cancer stage IV.

Embodiment 12: The method of any of embodiments 1-11, wherein selecting the pairwise subclass representative feature use of one or more of an information gain model, Random forest model, Anova model, correlation filtering model, or T-test model.

Embodiment 13: A method of designing a plurality of nucleic acid baits, comprising: generating a feature group according to any one of the methods of embodiments 1-3 or 6-12; designing a plurality of nucleic acid baits corresponding to genomic regions covering one or more features of the feature group.

Embodiment 14: A method of making a plurality of nucleic acid baits, comprising designing a feature group according to the method of embodiment 13; making the plurality of nucleic acid baits corresponding to genomic regions covering one or more features of the feature group.

Embodiment 15: A method of training a machine learning model to classify a test sample as a first disease characterization class or a second characterization class, comprising: receiving, at one or more processors, training data comprising values for features for a plurality of training samples, comprising a disease characterization subclass label, wherein the disease characterization subclass label represents one of a plurality of disease characterization subclasses the training sample belongs to and wherein the plurality of training samples represents a plurality of disease characterization subclass labels, and wherein the features comprise a plurality of features from a feature group generated according to the method of any one of embodiments 1-12; selecting, by one or more processors, a first subset of the plurality of the disease characterization subclass labels as belonging to the first disease characterization class and a second subset of the plurality of the disease characterization subclass labels as belonging to the second disease characterization class; training, by the one or more processors a machine learning model to classify a test sample as the first disease characterization class or the second disease characterization class using the training data, and the plurality of features from the feature group.

Embodiment 16: The method of embodiment 15, further comprising, performing a dimensionality reduction method on the feature group before training the machine learning model.

Embodiment 17: The method of embodiment 16, wherein the dimensionality reduction method comprises principal component analysis (PCA) or UMAP.

Embodiment 18: The method any of embodiments 15-17, wherein the machine learning model comprises one or more of a GLMnet, Random Forest, Support Vector Machine, Naïve Boost, Naïve Bayesian, XGboost, or Logistic regression model.

Embodiment 19: A method of classifying a test sample for a subject as a first disease characterization class or a second disease characterization class comprising: receiving, at one or more processors, test data from a test sample collected from the subject, test data comprising values for a plurality of features of a feature group generated according to the method of any one of embodiments 1-12; inputting, by the one or more processors, the test data into a machine learning model trained to classify a test sample as the first disease characterization class or the second disease characterization class according to the method of any one of embodiments 15-18, classifying the test sample as the first disease characterization class or the second disease characterization class based on the output from the classification model.

Embodiment 20: A method comprising: providing a plurality of nucleic acid molecules obtained from a test sample from a subject; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, using one or more processors, sequence read data for the plurality of sequence reads; generating, using the one or more processors, test data from the sequence read data, wherein the test data comprises values for features; inputting, by the one or more processors, the test data into a machine learning model trained to classify a test sample as the first disease characterization class or the second disease characterization class according to the method of any one of embodiments 15-18, classifying the test sample as the first disease characterization class or the second disease characterization class based on the output from the classification model.

Embodiment 21: The method of any one of embodiments 1-3 or 6-20, wherein the training data has been generated by: providing a plurality of nucleic acid molecules obtained from a training sample of the plurality of training samples; ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; sequencing, by a sequencer, the captured nucleic acid molecules to obtain a plurality of sequence reads that represent the captured nucleic acid molecules; receiving, using one or more processors, sequence read data for the plurality of sequence reads; generating, using the one or more processors, training data from the sequence read data, wherein the training data comprises values for features.

Embodiment 22: The method of any one of embodiments 19-21, wherein the subject is suspected of having or is determined to have cancer.

Embodiment 23: The method of embodiment 22, wherein the cancer is a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

Embodiment 24: The method of embodiment 23, wherein the cancer comprises acute lymphoblastic leukemia (Philadelphia chromosome positive), acute lymphoblastic leukemia (precursor B-cell), acute myeloid leukemia (FLT3+), acute myeloid leukemia (with an IDH2 mutation), anaplastic large cell lymphoma, basal cell carcinoma, B-cell chronic lymphocytic leukemia, bladder cancer, breast cancer (HER2 overexpressed/amplified), breast cancer (HER2+), breast cancer (HR+, HER2−), cervical cancer, cholangiocarcinoma, chronic lymphocytic leukemia, chronic lymphocytic leukemia (with 17p deletion), chronic myelogenous leukemia, chronic myelogenous leukemia (Philadelphia chromosome positive), classical Hodgkin lymphoma, colorectal cancer, colorectal cancer (dMMR/MSI-H), colorectal cancer (KRAS wild type), cryopyrin-associated periodic syndrome, a cutaneous T-cell lymphoma, dermatofibrosarcoma protuberans, a diffuse large B-cell lymphoma, fallopian tube cancer, a follicular B-cell non-Hodgkin lymphoma, a follicular lymphoma, gastric cancer, gastric cancer (HER2+), gastroesophageal junction (GEJ) adenocarcinoma, a gastrointestinal stromal tumor, a gastrointestinal stromal tumor (KIT+), a giant cell tumor of the bone, a glioblastoma, granulomatosis with polyangiitis, a head and neck squamous cell carcinoma, a hepatocellular carcinoma, Hodgkin lymphoma, juvenile idiopathic arthritis, lupus erythematosus, a mantle cell lymphoma, medullary thyroid cancer, melanoma, a melanoma with a BRAF V600 mutation, a melanoma with a BRAF V600E or V600K mutation, Merkel cell carcinoma, multicentric Castleman's disease, multiple hematologic malignancies including Philadelphia chromosome-positive ALL and CML, multiple myeloma, myelofibrosis, a non-Hodgkin's lymphoma, a nonresectable subependymal giant cell astrocytoma associated with tuberous sclerosis, a non-small cell lung cancer, a non-small cell lung cancer (ALK+), a non-small cell lung cancer (PD-L1+), a non-small cell lung cancer (with ALK fusion or ROS1 gene alteration), a non-small cell lung cancer (with BRAF V600E mutation), a non-small cell lung cancer (with an EGFR exon 19 deletion or exon 21 substitution (L858R) mutations), a non-small cell lung cancer (with an EGFR T790M mutation), ovarian cancer, ovarian cancer (with a BRCA mutation), pancreatic cancer, a pancreatic, gastrointestinal, or lung origin neuroendocrine tumor, a pediatric neuroblastoma, a peripheral T-cell lymphoma, peritoneal cancer, prostate cancer, a renal cell carcinoma, rheumatoid arthritis, a small lymphocytic lymphoma, a soft tissue sarcoma, a solid tumor (MSI-H/dMMR), a squamous cell cancer of the head and neck, a squamous non-small cell lung cancer, thyroid cancer, a thyroid carcinoma, urothelial cancer, a urothelial carcinoma, or Waldenstrom's macroglobulinemia.

Embodiment 25: The method of any of embodiments 22-24, further comprising treating the subject with an anti-cancer therapy.

Embodiment 26: The method of embodiment 25, wherein the anti-cancer therapy comprises a targeted anti-cancer therapy.

Embodiment 27: The method of embodiment 26, wherein the targeted anti-cancer therapy comprises abemaciclib (Verzenio), abiraterone acetate (Zytiga), acalabrutinib (Calquence), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), avapritinib (Ayvakit), avelumab (Bavencio), axicabtagene ciloleucel (Yescarta), axitinib (Inlyta), belantamab mafodotin-blmf (Blenrep), belimumab (Benlysta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib (Cabometyx), cabozantinib (Cabometyx, Cometriq), canakinumab (Ilaris), capmatinib hydrochloride (Tabrecta), carfilzomib (Kyprolis), cemiplimab-rwlc (Libtayo), ceritinib (LDK378/Zykadia), cetuximab (Erbitux), cobimetinib (Cotellic), crizotinib (Xalkori), dabrafenib (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib (Tarceva), everolimus (Afinitor), exemestane (Aromasin), fam-trastuzumab deruxtecan-nxki (Enhertu), fedratinib hydrochloride (Inrebic), fulvestrant (Faslodex), gefitinib (Iressa), gemtuzumab ozogamicin (Mylotarg), gilteritinib (Xospata), glasdegib maleate (Daurismo), hyaluronidase-zzxf (Phesgo), ibrutinib (Imbruvica), ibritumomab tiuxetan (Zevalin), idecabtagene vicleucel (Abecma), idelalisib (Zydelig), imatinib mesylate (Gleevec), infigratinib phosphate (Truseltiq), inotuzumab ozogamicin (Besponsa), ipilimumab (Yervoy), isatuximab-irfc (Sarclisa), ivosidenib (Tibsovo), ixazomib citrate (Ninlaro), lanreotide acetate (Somatuline Depot), lapatinib (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177-dotatate (Lutathera), margetuximab-cmkb (Margenza), midostaurin (Rydapt), mobocertinib succinate (Exkivity), mogamulizumab-kpkc (Poteligeo), moxetumomab pasudotox-tdfk (Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), obinutuzumab (Gazyva), ofatumumab (Arzerra), olaparib (Lynparza), olaratumab (Lartruvo), osimertinib (Tagrisso), palbociclib (Ibrance), panitumumab (Vectibix), pazopanib (Votrient), pembrolizumab (Keytruda), pemigatinib (Pemazyre), pertuzumab (Perjeta), pexidartinib hydrochloride (Turalio), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), ribociclib (Kisqali), ripretinib (Qinlock), rituximab (Rituxan), rituximab and hyaluronidase human (Rituxan Hycela), romidepsin (Istodax), rucaparib camsylate (Rubraca), ruxolitinib phosphate (Jakafi), sacituzumab govitecan-hziy (Trodelvy), seliciclib, selinexor (Xpovio), selpercatinib (Retevmo), selumetinib sulfate (Koselugo), siltuximab (Sylvant), sirolimus protein-bound particles (Fyarro), sonidegib (Odomzo), sorafenib (Nexavar), sotorasib (Lumakras), sunitinib (Sutent), tafasitamab-cxix (Monjuvi), tagraxofusp-erzs (Elzonris), talazoparib tosylate (Talzenna), tamoxifen (Nolvadex), tazemetostat hydrobromide (Tazverik), tebentafusp-tebn (Kimmtrak), temsirolimus (Torisel), tepotinib hydrochloride (Tepmetko), tisagenlecleucel (Kymriah), tisotumab vedotin-tftv (Tivdak), tocilizumab (Actemra), tofacitinib (Xeljanz), tositumomab (Bexxar), trametinib (Mekinist), trastuzumab (Herceptin), tretinoin (Vesanoid), tivozanib hydrochloride (Fotivda), toremifene (Fareston), tucatinib (Tukysa), umbralisib tosylate (Ukoniq), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap), or any combination thereof.

Embodiment 28: The method of any one of embodiments 1-27, further comprising obtaining the plurality of training samples from a plurality of training subjects.

Embodiment 29: The method of any one of embodiments 19-28, further comprising obtaining the test sample from the subject.

Embodiment 30: The method of any one of embodiments 1-29, wherein the plurality of training samples comprises a tissue biopsy sample, a liquid biopsy sample, or a normal control.

Embodiment 31: The method of any one of embodiments 19-30, wherein the test sample comprises a tissue biopsy sample, a liquid biopsy sample, or a normal control.

Embodiment 32: The method of embodiment 30 or 31, wherein the test sample and/or the plurality of training samples is a liquid biopsy sample and comprises blood, plasma, cerebrospinal fluid, sputum, stool, urine, or saliva.

Embodiment 33: The method of embodiment 32, wherein the test sample and/or the plurality of training samples is a liquid biopsy sample and comprises circulating tumor cells (CTCs).

Embodiment 34: The method of embodiment 32, wherein the test sample and/or plurality of training samples is a liquid biopsy sample and comprises cell-free DNA (cfDNA).

Embodiment 35: The method of embodiment 34, wherein the cell-free DNA (cfDNA) or a portion thereof comprises circulating tumor DNA (ctDNA).

Embodiment 36: The method of any one of embodiments 20-35, wherein the plurality of nucleic acid molecules comprises a mixture of tumor nucleic acid molecules and non-tumor nucleic acid molecules.

Embodiment 37: The method of embodiment 36, wherein the tumor nucleic acid molecules are derived from a tumor portion of a heterogeneous tissue biopsy sample, and the non-tumor nucleic acid molecules are derived from a normal portion of the heterogeneous tissue biopsy sample.

Embodiment 38: The method of embodiment 37, wherein the test sample and/or plurality of training samples comprises a liquid biopsy sample, and wherein the tumor nucleic acid molecules are derived from a circulating tumor DNA (ctDNA) fraction of the liquid biopsy sample, and the non-tumor nucleic acid molecules are derived from a non-tumor, cell-free DNA (cfDNA) fraction of the liquid biopsy sample.

Embodiment 39: The method of any one of embodiments 20-38, wherein the one or more adapters comprise amplification primers, flow cell adaptor sequences, substrate adapter sequences, or sample index sequences.

Embodiment 40: The method of any one of embodiments 20-39, wherein the captured nucleic acid molecules are captured from the amplified nucleic acid molecules by hybridization to one or more bait molecules.

Embodiment 41: The method of embodiment 40, wherein the one or more bait molecules comprise one or more nucleic acid molecules, each comprising a region that is complementary to a region of a captured nucleic acid molecule.

Embodiment 42: The method of any one of embodiments 20-41, wherein amplifying nucleic acid molecules comprises performing a polymerase chain reaction (PCR) amplification technique, a non-PCR amplification technique, or an isothermal amplification technique.

Embodiment 43: The method of any one of embodiments 20-42, wherein the sequencing comprises use of a massively parallel sequencing (MPS) technique, whole genome sequencing (WGS), whole exome sequencing, targeted sequencing, direct sequencing, or Sanger sequencing technique.

Embodiment 44: The method of embodiment 43, wherein the sequencing comprises massively parallel sequencing, and the massively parallel sequencing technique comprises next generation sequencing (NGS).

Embodiment 45: The method of any one of embodiments 20-44, wherein the sequencer comprises a next generation sequencer.

Embodiment 46: The method of any one of embodiments 20-45, wherein one or more of the plurality of sequencing reads overlap one or more gene loci within one or more subject intervals in the sample.

Embodiment 47: The method of embodiment 46, wherein the one or more gene loci comprises between 10 and 20 loci, between 10 and 40 loci, between 10 and 60 loci, between 10 and 80 loci, between 10 and 100 loci, between 10 and 150 loci, between 10 and 200 loci, between 10 and 250 loci, between 10 and 300 loci, between 10 and 350 loci, between 10 and 400 loci, between 10 and 450 loci, between 10 and 500 loci, between 20 and 40 loci, between 20 and 60 loci, between 20 and 80 loci, between 20 and 100 loci, between 20 and 150 loci, between 20 and 200 loci, between 20 and 250 loci, between 20 and 300 loci, between 20 and 350 loci, between 20 and 400 loci, between 20 and 500 loci, between 40 and 60 loci, between 40 and 80 loci, between 40 and 100 loci, between 40 and 150 loci, between 40 and 200 loci, between 40 and 250 loci, between 40 and 300 loci, between 40 and 350 loci, between 40 and 400 loci, between 40 and 500 loci, between 60 and 80 loci, between 60 and 100 loci, between 60 and 150 loci, between 60 and 200 loci, between 60 and 250 loci, between 60 and 300 loci, between 60 and 350 loci, between 60 and 400 loci, between 60 and 500 loci, between 80 and 100 loci, between 80 and 150 loci, between 80 and 200 loci, between 80 and 250 loci, between 80 and 300 loci, between 80 and 350 loci, between 80 and 400 loci, between 80 and 500 loci, between 100 and 150 loci, between 100 and 200 loci, between 100 and 250 loci, between 100 and 300 loci, between 100 and 350 loci, between 100 and 400 loci, between 100 and 500 loci, between 150 and 200 loci, between 150 and 250 loci, between 150 and 300 loci, between 150 and 350 loci, between 150 and 400 loci, between 150 and 500 loci, between 200 and 250 loci, between 200 and 300 loci, between 200 and 350 loci, between 200 and 400 loci, between 200 and 500 loci, between 250 and 300 loci, between 250 and 350 loci, between 250 and 400 loci, between 250 and 500 loci, between 300 and 350 loci, between 300 and 400 loci, between 300 and 500 loci, between 350 and 400 loci, between 350 and 500 loci, or between 400 and 500 loci.

Embodiment 48: The method of embodiment 46 or 47, wherein the one or more gene loci comprise ABL1, ACVRIB, AKT1, AKT2, AKT3, ALK, ALOX12B, AMER1, APC, AR, ARAF, ARFRP1, ARIDIA, ASXL1, ATM, ATR, ATRX, AURKA, AURKB, AXIN1, AXL, BAP1, BARD1, BCL2, BCL2L1, BCL2L2, BCL6, BCOR, BCORL1, BCR, BRAF, BRCA1, BRCA2, BRD4, BRIP1, BTG1, BTG2, BTK, CALR, CARD11, CASP8, CBFB, CBL, CCND1, CCND2, CCND3, CCNE1, CD22, CD274, CD70, CD74, CD79A, CD79B, CDC73, CDH1, CDK12, CDK4, CDK6, CDK8, CDKNIA, CDKNIB, CDKN2A, CDKN2B, CDKN2C, CEBPA, CHEK1, CHEK2, CIC, CREBBP, CRKL, CSFIR, CSF3R, CTCF, CTNNA1, CTNNB1, CUL3, CUL4A, CXCR4, CYP17A1, DAXX, DDR1, DDR2, DIS3, DNMT3A, DOTIL, EED, EGFR, EMSY (C11orf30), EP300, EPHA3, EPHB1, EPHB4, ERBB2, ERBB3, ERBB4, ERCC4, ERG, ERRFI1, ESR1, ETV4, ETV5, ETV6, EWSR1, EZH2, EZR, FAM46C, FANCA, FANCC, FANCG, FANCL, FAS, FBXW7, FGF10, FGF12, FGF14, FGF19, FGF23, FGF3, FGF4, FGF6, FGFR1, FGFR2, FGFR3, FGFR4, FH, FLCN, FLT1, FLT3, FOXL2, FUBP1, GABRA6, GATA3, GATA4, GATA6, GID4 (C17orf39), GNA11, GNA13, GNAQ, GNAS, GRM3, GSK3B, H3F3A, HDAC1, HGF, HNF1A, HRAS, HSD3B1, ID3, IDH1, IDH2, IGFIR, IKBKE, IKZF1, INPP4B, IRF2, IRF4, IRS2, JAK1, JAK2, JAK3, JUN, KDM5A, KDM5C, KDM6A, KDR, KEAP1, KEL, KIT, KLHL6, KMT2A (MLL), KMT2D (MLL2), KRAS, LTK, LYN, MAF, MAP2K1, MAP2K2, MAP2K4, MAP3K1, MAP3K13, MAPK1, MCL1, MDM2, MDM4, MED12, MEF2B, MEN1, MERTK, MET, MITF, MKNK1, MLH1, MPL, MRE11A, MSH2, MSH3, MSH6, MSTIR, MTAP, MTOR, MUTYH, MYB, MYC, MYCL, MYCN, MYD88, NBN, NF1, NF2, NFE2L2, NFKBIA, NKX2-1, NOTCH1, NOTCH2, NOTCH3, NPM1, NRAS, NT5C2, NTRK1, NTRK2, NTRK3, NUTM1, P2RY8, PALB2, PARK2, PARP1, PARP2, PARP3, PAX5, PBRM1, PDCD1, PDCDILG2, PDGFRA, PDGFRB, PDK1, PIK3C2B, PIK3C2G, PIK3CA, PIK3CB, PIK3R1, PIM1, PMS2, POLD1, POLE, PPARG, PPP2R1A, PPP2R2A, PRDM1, PRKARIA, PRKCI, PTCH1, PTEN, PTPN11, PTPRO, QKI, RAC1, RAD21, RAD51, RAD51B, RAD51C, RAD51D, RAD52, RAD54L, RAF1, RARA, RB1, RBM10, REL, RET, RICTOR, RNF43, ROS1, RPTOR, RSPO2, SDC4, SDHA, SDHB, SDHC, SDHD, SETD2, SF3B1, SGK1, SLC34A2, SMAD2, SMAD4, SMARCA4, SMARCB1, SMO, SNCAIP, SOCS1, SOX2, SOX9, SPEN, SPOP, SRC, STAG2, STAT3, STK11, SUFU, SYK, TBX3, TEK, TERC, TERT, TET2, TGFBR2, TIPARP, TMPRSS2, TNFAIP3, TNFRSF14, TP53, TSC1, TSC2, TYRO3, U2AF1, VEGFA, VHL, WHSC1, WHSCIL1, WT1, XPO1, XRCC2, ZNF217, ZNF703, or any combination thereof.

Embodiment 49: The method of embodiment 46 or 47, wherein the one or more gene loci comprise ABL, ALK, ALL, B4GALNT1, BAFF, BCL2, BRAF, BRCA, BTK, CD19, CD20, CD3, CD30, CD319, CD38, CD52, CDK4, CDK6, CML, CRACC, CS1, CTLA-4, dMMR, EGFR, ERBB1, ERBB2, FGFR1-3, FLT3, GD2, HDAC, HER1, HER2, HR, IDH2, IL-1β, IL-6, IL-6R, JAK1, JAK2, JAK3, KIT, KRAS, MEK, MET, MSI-H, mTOR, PARP, PD-1, PDGFR, PDGFRα, PDGFRβ, PD-L1, PI3Kδ, PIGF, PTCH, RAF, RANKL, RET, ROS1, SLAMF7, VEGF, VEGFA, VEGFB, or any combination thereof.

Embodiment 50: The method of any one of embodiments 19-49, further comprising generating, by the one or more processors, a report indicating the classification of the test sample as the first disease characterization class or the second disease characterization class.

Embodiment 51: The method of embodiment 50, further comprising transmitting the report to a healthcare provider.

Embodiment 52: The method of embodiment 51, wherein the report is transmitted via a computer network or a peer-to-peer connection.

Embodiment 53: The method of any one of embodiments 19-52, wherein the classification of the test sample as the first disease characterization class or the second disease characterized class is used to diagnose or confirm a diagnosis of a disease in the subject.

Embodiment 54: The method of embodiment 53, wherein the disease is cancer.

Embodiment 55: The method of embodiment 54, wherein the cancer is colorectal cancer.

Embodiment 56: The method of any of embodiments 19-55, further comprising selecting an anti-cancer therapy to administer to the subject based on the classification of the test sample.

Embodiment 57: The method of any one of embodiments 19-56, further comprising determining an effective amount of an anti-cancer therapy to administer to the subject based on the classification of the test sample.

Embodiment 58: The method of any one of embodiments 19-57, further comprising administering an anti-cancer therapy to the subject based on the classification of the test sample.

Embodiment 59: The method of any one of embodiments 56-58, wherein the anti-cancer therapy comprises chemotherapy, radiation therapy, immunotherapy, a targeted therapy, or surgery.

Embodiment 60: A method for diagnosing a disease, the method comprising: diagnosing that a subject has the disease based on a classification of a test sample from the subject as a first disease characterization class or a second disease characterization class, wherein the test sample is classified according to the method of any one of embodiments 19-59.

Embodiment 61: A method of selecting an anti-cancer therapy, the method comprising: classifying a test sample from the subject as a first disease characterization class or a second disease characterization class according to the methods of any one of embodiments 19-59; responsive to the classification of the test sample as the first disease characterization class or second disease characterization class, selecting an anti-cancer therapy for the subject.

Embodiment 62: A method of treating a cancer in a subject, comprising: classifying a test sample from the subject as a first disease characterization class or a second disease characterization class according to the methods of any one of embodiments 19-59; responsive to the classification of the test sample, administering an effective amount of an anti-cancer therapy to the subject.

Embodiment 63: A method for monitoring cancer progression or recurrence in a subject, the method comprising: classifying a first test sample from the subject as a first disease characterization class or a second disease characterization class according to the methods of any one of embodiments 19-59; determining a second classification in a second test sample obtained from the subject at a second time point; and comparing the first classification to the second classification, thereby monitoring the cancer progression or recurrence.

Embodiment 64: The method of embodiment 63, wherein the second classification for the second test sample is classified according to the method of any one of embodiment 19-59.

Embodiment 65: The method of embodiment 63 or 64, further comprising selecting an anti-cancer therapy for the subject in response to the cancer progression.

Embodiment 66: The method of embodiment 63 or 64, further comprising administering an anti-cancer therapy to the subject in response to the cancer progression.

Embodiment 67: The method of embodiment 63 or 64, further comprising adjusting an anti-cancer therapy for the subject in response to the cancer progression.

Embodiment 68: The method of any one of embodiments 63-67, further comprising adjusting a dosage of the anti-cancer therapy or selecting a different anti-cancer therapy in response to the cancer progression.

Embodiment 69: The method of embodiment 68, further comprising administering the adjusted anti-cancer therapy to the subject.

Embodiment 70: The method of any one of embodiments 63-69, wherein the first time point is before the subject has been administered an anti-cancer therapy, and wherein the second time point is after the subject has been administered the anti-cancer therapy.

Embodiment 71: The method of any one of embodiments 63-70, wherein the subject has a cancer, is at risk of having a cancer, is being routine tested for cancer, or is suspected of having a cancer.

Embodiment 72: The method of any one of embodiments 63-71, wherein the cancer is a solid tumor.

Embodiment 73: The method of any one of embodiments 63-72, wherein the cancer is a hematological cancer.

Embodiment 74: The method of any one of embodiments 65-73, wherein the anti-cancer therapy comprises chemotherapy, radiation therapy, immunotherapy, a targeted therapy, or surgery.

Embodiment 75: The method of any one of embodiments 19-59, further comprising determining, identifying, or applying the classification of the test sample from the subject as a diagnostic value associated with the test sample.

Embodiment 76: The method of any one of embodiments 19-59, further comprising generating a genomic profile for the subject based on the classification of the test sample.

Embodiment 77: The method of embodiment 76, wherein the genomic profile for the subject further comprises results from a comprehensive genomic profiling (CGP) test, a gene expression profiling test, a cancer hotspot panel test, a DNA methylation test, a DNA fragmentation test, an RNA fragmentation test, or any combination thereof.

Embodiment 78: The method of embodiment 76 or 77, wherein the genomic profile for the subject further comprises results from a nucleic acid sequencing-based test.

Embodiment 79: The method of any one of embodiments 76-78, further comprising selecting an anti-cancer therapy, administering an anti-cancer therapy, or applying an anti-cancer therapy to the subject based on the generated genomic profile.

Embodiment 80: The method of any one of embodiments 19-59, wherein the classification of the test sample as a first disease classification class and/or a second disease classification class is used in making suggested treatment decisions for the subject.

Embodiment 81: The method of any one of embodiments 19-59, wherein the classification of the test sample as a first disease classification class and/or a second disease classification class for the test sample is used in applying or administering a treatment to the subject.

Embodiment 82: A bait set comprising a plurality of nucleic acid baits made according to the method of embodiment 14.

Embodiment 83: A system comprising: one or more processors; and a memory communicatively coupled to the one or more processors and configured to store instructions that, when executed by the one or more processors, cause the system to: receive, at one or more processors, training data comprising values for features for a plurality of training samples comprising a disease characterization subclass label, wherein the disease characterization subclass label represents one of a plurality of disease characterization subclasses to which the training sample belongs to and wherein the plurality of training samples represents a plurality of disease characterization subclass labels; select, by the one or more processors, pairwise subclass representative features that distinguish the disease characterization subclasses from other disease characterization subclasses in the plurality of disease characterization subclasses; select, by one or more processors, a first subset of the plurality of the disease characterization subclass labels as belonging to the first disease characterization class and a second subset of the plurality of the disease characterization subclass labels as belonging to the second disease characterization class; select, by one or more processors, a subset of pairwise subclass representative features from the pairwise subclass representative features, wherein the subset of pairwise subclass representative features that distinguish disease characterization subclasses from within a common disease characterization class; generate, by the one or more processors, a feature group by removing the subset of pairwise subclass representative features from the pairwise subclass representative features.

Embodiment 84: A non-transitory computer-readable storage medium storing one or more programs, the one or more programs comprising instructions, which when executed by one or more processors of a system, cause the system to: receive, at one or more processors, training data comprising values for features for a plurality of training samples comprising a disease characterization subclass label, wherein the disease characterization subclass label represents one of a plurality of disease characterization subclasses to which the training sample belongs to and wherein the plurality of training samples represents a plurality of disease characterization subclass labels; select, by the one or more processors, pairwise subclass representative features that distinguish the disease characterization subclasses from other disease characterization subclasses in the plurality of disease characterization subclasses; select, by one or more processors, a first subset of the plurality of the disease characterization subclass labels as belonging to the first disease characterization class and a second subset of the plurality of the disease characterization subclass labels as belonging to the second disease characterization class; select, by one or more processors, a subset of pairwise subclass representative features from the pairwise subclass representative features, wherein the subset of pairwise subclass representative features that distinguish disease characterization subclasses from within a common disease characterization class; generate, by the one or more processors, a feature group by removing the subset of pairwise subclass representative features from the pairwise subclass representative features.

It should be understood from the foregoing that, while particular implementations of the disclosed methods and systems have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A method of generating a feature group for distinguishing a first disease characterization class from a second disease characterization class, comprising:
receiving, at one or more processors, training data comprising values for features for a plurality of training samples comprising a disease characterization subclass label representing one of a plurality of disease characterization subclasses to which a corresponding training sample belongs, wherein the plurality of training samples represents a plurality of disease characterization subclass labels;
selecting, by the one or more processors, pairwise subclass representative features that distinguish the disease characterization subclasses from other disease characterization subclasses in the plurality of disease characterization subclasses;
selecting, by one or more processors, a first subset of the plurality of the disease characterization subclass labels as belonging to the first disease characterization class and a second subset of the plurality of the disease characterization subclass labels as belonging to the second disease characterization class;
selecting, by one or more processors, a subset of pairwise subclass representative features from the pairwise subclass representative features, wherein the subset of pairwise subclass representative features comprises features that distinguish disease characterization subclasses from within a common disease characterization class;
generating, by the one or more processors, a feature group by removing the subset of pairwise subclass representative features from the pairwise subclass representative features.

2. The method of claim 1, wherein the features are associated with a plurality of subject intervals, a plurality of genes, a plurality of epigenetic states and/or a plurality of genetic alterations.

3. The method of claim 1, wherein the values comprise mRNA expression values, protein expression values, methylation levels, and/or a genetic alteration status.

4. The method of claim 1, wherein the features are associated with a plurality of microbacterium species.

5. The method of claim 4, wherein the values comprise an abundance of a microbacterium species in the plurality of microbacterium species.

6. The method of claim 1, wherein the disease characterization subclasses relate to a pathological status, tissue of origin, or stage of a disease.

7. The method of claim 1, wherein the first disease characterization class and/or second disease characterization class comprises a cancer characterization.

8. The method of claim 1, wherein the first disease characterization class is cancer, and the second disease characterization class is non-cancer.

9. The method of claim 8, wherein the first subset of disease characterization subclasses comprises, colorectal cancer, advanced adenoma, lung cancer, lung adenocarcinoma, lung squamous-cell carcinoma, esophageal cancer, stomach cancer, liver cancer, bile duct cancer, pancreatic cancer, bladder cancer, and ovarian cancer, and the second subset of disease characterization subclasses comprises non-advanced adenoma and non-cancer.

10. The method of claim 1, wherein the first disease characterization class is colorectal cancer, and the second disease characterization class is lung cancer.

11. The method of claim 10, wherein the first subset of disease characterization subclasses comprises, colorectal cancer stage I, colorectal cancer stage II, colorectal cancer stage III, colorectal cancer stage IV, and the second subset of disease characterization subclasses comprises lung cancer stage I, lung cancer stage II, lung cancer stage III, lung cancer stage IV.

12. The method of claim 1, comprising training a machine learning model based on the feature group.

13. A method of training a machine learning model to classify a test sample as a first disease characterization class or a second disease characterization class, comprising:
receiving, at one or more processors, training data comprising values for features for a plurality of training samples, comprising a disease characterization subclass label,
wherein the disease characterization subclass label represents one of a plurality of disease characterization subclasses the training sample belongs to and wherein the plurality of training samples represents a plurality of disease characterization subclass labels, and wherein the features comprise a plurality of features from a feature group generated according to the method of claim 1;
selecting, by one or more processors, a first subset of the plurality of the disease characterization subclass labels as belonging to the first disease characterization class and a second subset of the plurality of the disease characterization subclass labels as belonging to the second disease characterization class;
training, by the one or more processors, a machine learning model to classify a test sample as the first disease characterization class or the second disease characterization class using the training data, and the plurality of features from the feature group.

14. The method of claim 13, further comprising, performing a dimensionality reduction method on the feature group before training the machine learning model.

15. A method of classifying a test sample for a subject as a first disease characterization class or a second disease characterization class, comprising:
receiving, at one or more processors, test data from a test sample collected from the subject, test data comprising values for a plurality of features of a feature group generated according to the method of claim 1;
inputting, by the one or more processors, the test data into a machine learning model trained to classify a test sample as the first disease characterization class or the second disease characterization class,
classifying the test sample as the first disease characterization class or the second disease characterization class based on the output from the classification model.

16. The method of claim 15, wherein the subject is suspected of having or is determined to have cancer.

17. The method of claim 15, further comprising generating, by the one or more processors, a report indicating the classification of the test sample as the first disease characterization class or the second disease characterization class.

18. The method of any claim 15, wherein the classification of the test sample as the first disease characterization class or the second disease characterization class is used to diagnose or confirm a diagnosis of a disease in the subject.

19. The method of claim 15, further comprising selecting an anti-cancer therapy to administer to the subject based on the classification of the test sample or administering an anti-cancer therapy to the subject based on the classification of the test sample.

20. A system comprising:
one or more processors; and
a memory communicatively coupled to the one or more processors and configured to store instructions that, when executed by the one or more processors, cause the system to:
receive, at one or more processors, training data comprising values for features for a plurality of training samples comprising a disease characterization subclass label representing one of a plurality of disease characterization subclasses to which the corresponding training sample belongs, wherein the plurality of training samples represents a plurality of disease characterization subclass labels;
select, by the one or more processors, pairwise subclass representative features that distinguish the disease characterization subclasses from other disease characterization subclasses in the plurality of disease characterization subclasses;
select, by one or more processors, a first subset of the plurality of the disease characterization subclass labels as belonging to the first disease characterization class and a second subset of the plurality of the disease characterization subclass labels as belonging to the second disease characterization class;
select, by one or more processors, a subset of pairwise subclass representative features from the pairwise subclass representative features comprises features, wherein the subset of pairwise subclass representative features that distinguish disease characterization subclasses from within a common disease characterization class;
generate, by the one or more processors, a feature group by removing the subset of pairwise subclass representative features from the pairwise subclass representative features.
